(19) United States
(12) Patent Application Publication
(10) Pub. No.: US 2017/0145506 A1
(43) Pub. Date: May 25, 2017

(54) Signature of Longitudinal Gene Expression Changes to Diagnose Brain Injury

(71) Applicant: University of Rochester, Rochester, NY (US)

(72) Inventors: Kian Merchant-Borna, Rochester, NY (US); Jeffrey Bazarian, Honeoye Falls, NY (US)

(21) Appl. No.: 15/360,421

(22) Filed: Nov. 23, 2016

Related U.S. Application Data
(60) Provisional application No. 62/259,302, filed on Nov. 24, 2015.

(51) Int. Cl. C12Q 1/68

(52) U.S. Cl. CPC C12Q 1/6883; C12Q 2600/158; C12Q 2600/16; C12Q 2600/118

(57) ABSTRACT

Described herein are compositions and methods relating to biomarkers used to diagnose brain injury in a subject who has experienced a head trauma. The biomarkers described herein can be used to diagnose, monitor the onset, monitor the progression, and assess the recovery of brain injury. The biomarkers can also be used to establish and evaluate treatment plans.

### CROSS-REFERENCE TO RELATED APPLICATIONS

The present application claims priority to U.S. Provisional Patent Application No. 62/259,302, filed Nov. 24, 2015, which is hereby incorporated herein by reference in its entirety.

### STATEMENT REGARDING FEDERALLY SPONSORED RESEARCH OR DEVELOPMENT

This invention was made with government support under K24HD064754 awarded by the National Institute of Health. The government has certain rights in the invention.

### BACKGROUND OF THE INVENTION

Despite 3.8 million sports-related concussions (SRC) in the United States annually, there is currently no approved treatment. This may be due in part to a limited understanding of concussion pathophysiology, as well as an inability to determine individuals at risk for long-term deficits (Langlois et al., 2006, J Head Trauma Rehabil, 21: 375-378). Global gene expression analysis using mRNA samples can provide valuable insights into the underlying pathophysiology and potential repair mechanisms of acute traumatic brain injury (TBI), including SRC. Gene expression changes have been previously characterized among severe TBI patients using post-mortem and post-operative brain tissue samples (Michael et al., 2005, Journal of Clinical Neuroscience, 12: 284-290; Liu et al., 2013, Neurological Sciences, 34: 1173-1180; Staffa et al., 2012, J Neurotrauma, 29: 2716-2721). However, little is known regarding gene expression in mild TBI (mTBI) and the signal present in blood. After severe TBI, differentially expressed genes were found to be related primarily to transcriptional regulation, energy metabolism, signal transduction, inflammation, and intercellular adhesion (Michael et al., 2005, Journal of Clinical Neuroscience, 12: 284-290). Although differential gene expression after less severe forms of TBI such as SRC has not been described, polymorphisms in several genes have been shown to influence outcome after non-sports related concussions. These genes include the calcium channel subunit (CACNA1A), brain derived neurotrophic factor (BDNF), dopamine D2 receptor (DRD2), dopamine active transporter (DAT), and dopamine β-hydroxylase (DBH) (McAllister, 2010, Pm & R, 2: S241-S252). While these polymorphisms could potentially be used to identify athletes at risk for poor outcome after SRC, it is unclear how they could be used to develop therapeutics for SRC.

A better understanding of the transcriptional and translational changes occurring in the brain after SRC is more likely to identify potential therapeutic targets. Progress on this front has been hampered by the inaccessibility of human brain tissue after SRC, as the mortality and need for neurosurgery from this injury is close to zero. In order to describe transcriptional changes after SRC, thus, less invasive approaches are needed. In 2006, Sullivan et al. demonstrated that gene expression changes in peripheral blood mononuclear cells (PBMCs) correlated with gene expression changes in the brain (Sullivan et al., 2006, American Journal of Medical Genetics Part B, Neuropsychiatric Genetics: the Official Publication of the International Society of Psychiatric Genetics, 141B: 261-268; Zhao et al., 2013, Journal of Alzheimer's Disease, 34: 417-429). Since that time, researchers have successfully used peripheral gene expression changes to understand the pathophysiology of neurologic disorders such as autism, schizophrenia, and posttraumatic stress disorder to suggest central mechanisms underlying symptoms (Segman et al., 2005, Molecular Psychiatry, 10: 500-513; Yehuda et al., 2009, Biological Psychiatry, 66: 708-711; Glatt et al., 2013 American Journal of Medical Genetics Part B, Neuropsychiatric Genetics: the Official Publication of the International Society of Psychiatric Genetics, 162B: 313-326). In the context of SRC, changes in PBMC gene expression profile may provide an ideal, clinically accessible window into the human brain by reflecting post-injury molecular alterations.

However, there remains a need in the art for compositions and methods for the diagnosis and treatment of SRC and TBI. The present invention satisfies this need.

### SUMMARY OF THE INVENTION

In one aspect, the present invention provides a method of diagnosing brain injury in a subject who has received a head trauma. In one embodiment, the method comprises detecting the level of at least one biomarker in a first biological sample obtained from the subject at a first time point following head trauma; determining that the level of the at least one biomarker in the first biological sample is different when compared to a control; and determining that the subject has a brain injury when the level of the at least one biomarker in the first biological sample is different when compared to the control; wherein the at least one biomarker is a gene or gene product listed in Table 4A, Table 4B, Table 5A, Table 5B, Table 6, Table 7A, Table 7B, Table 8A, Table 8B, Table 9A, or Table 9B.

In one embodiment, the control is the level of the at least one biomarker in a biological sample obtained from the subject prior to head trauma. In one embodiment, the control is the level or change in the level of the at least one biomarker in a control subject or population who have not experienced a head trauma. In one embodiment, the level of the at least one biomarker in the first biological sample is different from the control by more than about 1.5 fold.

In one embodiment, one or more of the at least one biomarker is a gene or gene product listed in Table 4A or Table 7A and the first time point is about 6 hours, and wherein the expression level of the at least one biomarker is increased compared to the control. In one embodiment, one or more of the at least one biomarker is a gene or gene product listed in Table 4B or Table 7B and the first time point is about 6 hours, and wherein the expression level of the at least one biomarker is decreased compared to the control.

In one embodiment, one or more of the at least one biomarker is a gene or gene product listed in Table 5A or Table 8A and the first time point is about 7 days, and wherein the expression level of the at least one biomarker is increased compared to the control. In one embodiment, one or more of the at least one biomarker is a gene or gene product listed in Table 5B or Table 8B and the first time point is about 7 days, and wherein the expression level of the at least one biomarker is decreased compared to the control.

In one embodiment, the at least one biomarker comprises an mRNA biomarker. In one embodiment, the at least one marker is a protein biomarker. In one embodiment, the first biological sample is a peripheral mononuclear blood cell (PMBC). In one embodiment, the method further comprises effectuating a brain injury treatment to the subject.

In one embodiment, the method comprises detecting the level of at least one biomarker in a first biological sample obtained from the subject at a first time point following head trauma; detecting the level of the at least one biomarker in a second biological sample obtained from the subject at a second time point following head trauma; determining that the level of the at least one biomarker in the second biological sample is different as compared to the level of the at least one biomarker in the first biological sample; and determining that the subject has a concussion when the level of the at least one biomarker in the second biological sample is different than the level of the at least one biomarker in the first biological sample; wherein the at least one biomarker is a gene or gene product listed in Table 6, Table 9A, or Table 9B.

In one embodiment, the first time point is about 6 hours following head trauma. In one embodiment, the second time point is about 7 days following head trauma.

In one embodiment, the method further comprises detecting that the difference in the level of the at least one biomarker in the second biological sample as compared to the level of the at least one biomarker in the first biological sample is different relative to a control.

In one embodiment, the at least one biomarker comprises an mRNA biomarker. In one embodiment, the at least one marker is a protein biomarker. In one embodiment, the first biological sample and second biological sample each comprise a peripheral mononuclear blood cell (PMBC). In one embodiment, the method further comprises effectuating a brain injury treatment to the subject.

In one aspect, the present invention relates to a method of assessing the recovery from brain injury in a subject who has received a head trauma. In one embodiment, the method comprises detecting the level of at least one biomarker in a first biological sample obtained from the subject at a first time point following head trauma; determining that the level of the at least one biomarker in the first biological sample is different as compared to a control; and determining the recovery from brain injury when the level of the at least one biomarker in the first biological sample is significantly different when compared to the control level; wherein the at least one biomarker is a gene or gene product listed in Table 4A, Table 4B, Table 5A, Table 5B, Table 6, Table 7A, Table 7B, Table 8A, Table 8B, Table 9A, or Table 9B.

In one embodiment, the method comprises detecting the level of at least one biomarker in a first biological sample obtained from the subject at a first time point following head trauma; detecting the level of the at least one biomarker in a second biological sample obtained from the subject at a second time point following head trauma; determining that the level of the at least one biomarker in the second biological sample is different as compared to the level of the at least one biomarker in the first biological sample; and determining the recovery from brain injury when the level of the at least one biomarker in the second biological sample is significantly different than the level of the at least one biomarker in the first biological sample; wherein the at least one biomarker is a gene or gene product listed in Table 6, Table 9A, or Table 9B.

In one aspect, the present invention provides a method of treating an individual with brain injury comprising administering a brain injury treatment to a subject identified as having a differentially expressed level of at least one biomarker in a biological sample obtained after head trauma, wherein the at least one biomarker is a gene or gene product listed in Table 4A, Table 4B, Table 5A, Table 5B, Table 6, Table 7A, Table 7B, Table 8A, Table 8B, Table 9A, or Table 9B.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is a schematic depicting the study design and analysis plan. Among athletes who suffered a SRC, gene expression was compared at baseline to acutely (within 6 hours) post-SRC (a), and at baseline to sub-acutely (7 days) post-SRC (b). Among uninjured teammate athletes (controls), a proiri analysis involved a comparison of gene expression at baseline to the same acute post-SRC time point as the injured athlete to whom they were matched (c). Because none of the baseline samples from uninjured control athletes were suitable for mRNA analysis, baseline samples from athletes who subsequently suffered a SRC (i.e., before they were injured) were used as a surrogate for uninjured control baseline mRNA expression (c*). Based on the assumption that mRNA expression among uninjured teammate controls would not change significantly over the 6 days spanning the acute-to-sub-acute time period, mRNA expression at the acute time point was applied to the sub-acute time point.

FIG. 2 is a schematic illustrating the number of probesets with significant changes in gene expression from baseline to post-SRC. Of the 54,675 total probesets, 766 were found to have significant changes in expression level from baseline to post-SRC. Specifically, 287 probesets were unique to (a) acutely after SRC, whereas 170 were unique to (c) sub-acutely after SRC. There were 309 probesets that were significantly differentially expressed from baseline to (b) both acutely and sub-acutely after SRC.

FIG. 3 is a heat map of differential gene expression among athletes before (baseline) and after (acute and sub-acute) SRC. Color-coded expression levels of the 766 probesets that were significantly changed post-SRC (acute and sub-acute) relative to baseline (X-axis) among 16 SRC athletes, standardized to mean 0 and standard deviation of 1. Up-regulated genes are red, down-regulated genes are blue, genes with unchanged expression are colored grey. Individual SRC athletes are along the Y-axis, and grouped by the 3 indicated time points. (There are fewer subjects at the acute time point because of inadequate RNA in 6 PBMC samples). A heat map of differential gene expression among uninjured athlete controls was not displayed because no significant pre-post changes in gene expression were detected.

FIG. 4 is a schematic illustrating the top network of differentially expressed genes acutely (within 6 hours) after SRC. Functional analysis of the top selected genes identified by microarray within 6 hours of SRC centered on the ‘Inflammatory Response, Infectious Disease, Renal and Urological Disease’ network. The network is graphically represented as nodes (genes) and lines (the biological relationship between genes). Red and green shaded nodes represent up- and down-regulated genes, respectively; empty nodes are those that are biologically linked to differentially expressed genes based on the evidence in the literature, but not differentially expressed in the analyzed samples. Solid lines represent a direct interaction between the two gene products while dotted lines indicates indirect interactions. Network hubs and their connections to each other are noted in bold. Only those genes with direct or indirect connections to one of the hubs were displayed in this figure for simplicity.

FIG. 5 is a schematic illustrating the top network of differentially expressed genes sub-acutely (at 7 days) after SRC. Functional analysis of the top selected genes identified by microarray within 7 days after SRC centered on the ‘Neurological Disease, Cell Death and Survival, Cell Cycle’ network. The network is graphically represented as nodes (genes) and lines (the biological relationship between genes). Red and green shaded nodes represent up- and down-regulated genes, respectively; empty nodes are those that are biologically linked to differentially expressed genes based on the evidence in the literature, but not differentially expressed in the analyzed samples. Solid lines represent a direct interaction between the two gene products while dotted lines indicate indirect interactions. Network hubs and their connections to each other are noted in bold. Only those genes with direct or indirect connections to one of the hubs were displayed in this figure for simplicity.

FIG. 6 is a schematic depicting the study design and analysis plan for experiments investigating the gene expression changes between a concussed twin and control twin.

FIG. 7, comprising FIG. 7A and FIG. 7B, is a set of graphs depicting the results of experiments, demonstrating the subject-specific expression changes of genes that exceeded a 1.5 fold difference between individuals.

### DETAILED DESCRIPTION

The present invention provides compositions and methods relating to biomarkers that can be used for the diagnoses of concussion or brain injury in a subject. The markers of the invention can be used to screen, diagnose, monitor the onset, monitor the progression, and assess the recovery of concussion or brain injury. The markers of the invention can be used to establish and evaluate treatment plans.

The present invention therefore provides compositions and methods of diagnosing and providing a prognosis for brain injury, such as concussion, sports related concussion (SRC), mild traumatic brain injury (mTBI), and the like. In certain embodiments, the method comprises examining relevant biomarkers and their expression. In one embodiment, biomarker expression includes transcription into messenger RNA (mRNA) and translation into protein. In certain embodiments, the method comprises determining if the expression levels of the relevant biomarkers are differentially expressed as compared to a control. In certain embodiments, the control may be the level of the relevant biomarkers in a subject not having a brain injury, a population not having a brain injury, a subject who has not recovered from a brain injury, a population that has not recovered from a brain injury, a subject that has recovered from a brain injury, a population that has recovered from a brain injury, and a control sample of the subject being diagnosed where the control sample is obtained prior to head trauma. In certain embodiments, the method comprises determining if the expression levels of the relevant biomarkers in a sample obtained from the subject are differentially expressed as compared to the expression levels of the relevant biomarkers in an earlier obtained sample from the subject, which was obtained at an earlier time point following brain trauma. In certain embodiments, the method comprises detecting the expression levels of the relevant biomarkers across a plurality of samples obtained from the subject overtime, thereby providing a timecourse of biomarker expression.

In one embodiment, the invention provides a biomarker for the detection of brain injury in a subject. In one embodiment, the biomarker for the detection of brain injury includes but is not limited to the biomarkers listed in Table 4A, Table 4B, Table 5A, Table 5B, Table 6, Table 7A, Table 7B, Table 8A, Table 8B, Table 9A, and Table 9B.

In one embodiment, the invention provides a biomarker for the detection of recovery from brain injury in a subject. In one embodiment, the biomarker for the detection of recovery from brain injury includes but is not limited to the biomarkers listed in Table 4A, Table 4B, Table 5A, Table 5B, Table 6, Table 7A, Table 7B, Table 8A, Table 8B, Table 9A, and Table 9B.

Accordingly, in some embodiments of the invention, methods for diagnosing brain injury are provided. The methods comprise a) providing a biological sample from the subject; b) analyzing the biological sample with an assay that specifically detects at least one biomarker of the invention in the biological sample; c) comparing the level of the at least one biomarker in the sample with the level in a control sample or earlier obtained biological sample, wherein a statistically significant difference between the level of the at least one biomarker in the sample with the level in a control sample or earlier obtained biological sample is indicative of brain injury. In some embodiments, the methods further comprise the step of d) effectuating a treatment regimen based thereon. In certain embodiments, the method comprises analyzing the change in gene expression over a defined time interval in the subject, and comparing detected change in gene expression with the change in gene expression observed in a control subject.

In some embodiments of the invention, methods for determining the recovery of brain injury are provided. The methods comprise a) providing a biological sample from the subject; b) analyzing the biological sample with an assay that specifically detects at least one biomarker of the invention in the biological sample; c) comparing the level of the at least one biomarker in the sample with the level in a control sample or earlier obtained biological sample, wherein a statistically significant difference between the level of the at least one biomarker in the sample with the level in a control sample or earlier obtained biological sample is indicative of recovery from brain injury. In some embodiments, the methods further comprise the step of d) effectuating a treatment regimen based thereon. In certain embodiments, the method comprises analyzing the change in gene expression over a defined time interval in the subject, and comparing detected change in gene expression with the change in gene expression observed in a control subject.

In one embodiment, the biomarker types comprise mRNA biomarkers. In various embodiments, the mRNA is detected by at least one of mass spectroscopy, PCR microarray, thermal sequencing, capillary array sequencing, solid phase sequencing, and the like.

In another embodiment, the biomarker types comprise polypeptide biomarkers. In various embodiments, the polypeptide is detected by at least one of ELISA, Western blot, flow cytometry, immunofluorescence, immunohistochemistry, mass spectroscopy, and the like.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

The term “amplification” refers to the operation by which the number of copies of a target nucleotide sequence present in a sample is multiplied.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. The antibodies in the present invention may exist in a variety of forms including, for example, polyclonal antibodies, monoclonal antibodies, intracellular antibodies (“intrabodies”), Fv, Fab and F(ab)2, as well as single chain antibodies (scFv), heavy chain antibodies, such as camelid antibodies, synthetic antibodies, chimeric antibodies, and humanized antibodies (Harlow et al., 1999, Using Antibodies: A Laboratory Manual, Cold Spring Harbor Laboratory Press, N.Y.; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. κ and λ light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

As used herein, an “immunoassay” refers to any binding assay that uses an antibody capable of binding specifically to a target molecule to detect and quantify the target molecule.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “coding sequence,” as used herein, means a sequence of a nucleic acid or its complement, or a part thereof, that can be transcribed and/or translated to produce the mRNA and/or the polypeptide or a fragment thereof. Coding sequences include exons in a genomic DNA or immature primary RNA transcripts, which are joined together by the cell's biochemical machinery to provide a mature mRNA. The anti-sense strand is the complement of such a nucleic acid, and the coding sequence can be deduced therefrom. In contrast, the term “non-coding sequence,” as used herein, means a sequence of a nucleic acid or its complement, or a part thereof, that is not translated into amino acid in vivo, or where tRNA does not interact to place or attempt to place an amino acid. Non-coding sequences include both intron sequences in genomic DNA or immature primary RNA transcripts, and gene-associated sequences such as promoters, enhancers, silencers, and the like.

As used herein, the terms “complementary” or “complementarity” are used in reference to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

As used herein, the term “diagnosis” refers to the determination of the presence of a disease or disorder. In some embodiments of the present invention, methods for making a diagnosis are provided which permit determination of the presence of a particular disease or disorder.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the Tₘ of the formed hybrid, and the G:C ratio within the nucleic acids. A single molecule that contains pairing of complementary nucleic acids within its structure is said to be “self-hybridized.” A single DNA molecule with internal complementarity could assume a variety of secondary structures including loops, kinks or, for long stretches of base pairs, coils.

“Instructional material,” as that term is used herein, includes a publication, a recording, a diagram, or any other medium of expression which can be used to communicate the usefulness of the nucleic acid, peptide, and/or compound of the invention in the kit for identifying, diagnosing or alleviating or treating the various diseases or disorders recited herein. Optionally, or alternately, the instructional material may describe one or more methods of identifying, diagnosing or alleviating the diseases or disorders in a cell or a tissue of a subject. The instructional material of the kit may, for example, be affixed to a container that contains one or more components of the invention or be shipped together with a container that contains the one or more components of the invention. Alternatively, the instructional material may be shipped separately from the container with the intention that the recipient uses the instructional material and the components cooperatively.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “label” when used herein refers to a detectable compound or composition that is conjugated directly or indirectly to a probe to generate a “labeled” probe. The label may be detectable by itself (e.g. radioisotope labels or fluorescent labels) or, in the case of an enzymatic label, may catalyze chemical alteration of a substrate compound or composition that is detectable (e.g., avidin-biotin). In some instances, primers can be labeled to detect a PCR product.

The terms “microarray” and “array” refers broadly to “DNA microarrays,” “DNA chip(s),” “protein microarrays” and “protein chip(s)” and encompasses all art-recognized solid supports, and all art-recognized methods for affixing nucleic acid, peptide, and polypeptide molecules thereto. Preferred arrays typically comprise a plurality of different nucleic acid or peptide probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 5,800,992, 6,040,193, 5,424,186 and Fodor et al., 1991, Science, 251:767-777, each of which is incorporated by reference in its entirety for all purposes. Arrays may generally be produced using a variety of techniques, such as mechanical synthesis methods or light directed synthesis methods that incorporate a combination of photolithographic methods and solid phase synthesis methods. Techniques for the synthesis of these arrays using mechanical synthesis methods are described in, e.g., U.S. Pat. Nos. 5,384,261, and 6,040,193, which are incorporated herein by reference in their entirety for all purposes. Although a planar array surface is preferred, the array may be fabricated on a surface of virtually any shape or even a multiplicity of surfaces. Arrays may be nucleic acids on beads, gels, polymeric surfaces, fibers such as fiber optics, glass or any other appropriate substrate. (See U.S. Pat. Nos. 5,770,358, 5,789,162, 5,708,153, 6,040,193 and 5,800,992, which are hereby incorporated by reference in their entirety for all purposes.) Arrays may be packaged in such a manner as to allow for diagnostic use or can be an all-inclusive device; e.g., U.S. Pat. Nos. 5,856,174 and 5,922,591 incorporated in their entirety by reference for all purposes. Arrays are commercially available from, for example, Affymetrix (Santa Clara, Calif.) and Applied Biosystems (Foster City, Calif.), and are directed to a variety of purposes, including genotyping, diagnostics, mutation analysis, marker expression, and gene expression monitoring for a variety of eukaryotic and prokaryotic organisms. The number of probes on a solid support may be varied by changing the size of the individual features. In one embodiment the feature size is 20 by 25 microns square, in other embodiments features may be, for example, 8 by 8, 5 by 5 or 3 by 3 microns square, resulting in about 2,600,000, 6,600,000 or 18,000,000 individual probe features.

Assays for amplification of the known sequence are also disclosed. For example primers for PCR may be designed to amplify regions of the sequence. For RNA, a first reverse transcriptase step may be used to generate double stranded DNA from the single stranded RNA. The array may be designed to detect sequences from an entire genome; or one or more regions of a genome, for example, selected regions of a genome such as those coding for a protein or RNA of interest; or a conserved region from multiple genomes; or multiple genomes, arrays and methods of genetic analysis using arrays is described in Cutler, et al., 2001, Genome Res. 11(11): 1913-1925 and Warrington, et al., 2002, Hum Mutat 19:402-409 and in US Patent Pub No 20030124539, each of which is incorporated herein by reference in its entirety.

A “nucleic acid” refers to a polynucleotide and includes poly-ribonucleotides and poly-deoxyribonucleotides. Nucleic acids according to the present invention may include any polymer or oligomer of pyrimidine and purine bases, preferably cytosine, thymine, and uracil, and adenine and guanine, respectively. (See Albert L. Lehninger, Principles of Biochemistry, at 793-800 (Worth Pub. 1982) which is herein incorporated in its entirety for all purposes). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferably at least 8, 15 or 25 nucleotides in length, but may be up to 50, 100, 1000, or 5000 nucleotides long or a compound that specifically hybridizes to a polynucleotide. Polynucleotides include sequences of deoxyribonucleic acid (DNA) or ribonucleic acid (RNA) or mimetics thereof which may be isolated from natural sources, recombinantly produced or artificially synthesized. A further example of a polynucleotide of the present invention may be a peptide nucleic acid (PNA). (See U.S. Pat. No. 6,156,501 which is hereby incorporated by reference in its entirety.) The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this disclosure. It will be understood that when a nucleotide sequence is represented herein by a DNA sequence (e.g., A, T, G, and C), this also includes the corresponding RNA sequence (e.g., A, U, G, C) in which “U” replaces “T”.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the term “polymerase chain reaction” (“PCR”) refers to the method of K. B. Mullis (U.S. Pat. Nos. 4,683,195 4,683,202, and 4,965,188, hereby incorporated by reference), which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”. As used herein, the terms “PCR product,” “PCR fragment,” “amplification product” or “amplicon” refer to the resultant mixture of compounds after two or more cycles of the PCR steps of denaturation, annealing and extension are complete. These terms encompass the case where there has been amplification of one or more segments of one or more target sequences.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, “polynucleotide” includes cDNA, RNA, DNA/RNA hybrid, antisense RNA, ribozyme, genomic DNA, synthetic forms, and mixed polymers, both sense and antisense strands, and may be chemically or biochemically modified to contain non-natural or derivatized, synthetic, or semi-synthetic nucleotide bases. Also, contemplated are alterations of a wild type or synthetic gene, including but not limited to deletion, insertion, substitution of one or more nucleotides, or fusion to other polynucleotide sequences.

The term “primer” refers to an oligonucleotide capable of acting as a point of initiation of synthesis along a complementary strand when conditions are suitable for synthesis of a primer extension product. The synthesizing conditions include the presence of four different deoxyribonucleotide triphosphates and at least one polymerization-inducing agent such as reverse transcriptase or DNA polymerase. These are present in a suitable buffer, which may include constituents which are co-factors or which affect conditions such as pH and the like at various suitable temperatures. A primer is preferably a single strand sequence, such that amplification efficiency is optimized, but double stranded sequences can be utilized.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention relates to compositions and methods for diagnosing the presence of brain injury or recovery from a brain injury. The compositions and methods may be used to assess exemplary injuries, including but not limited to concussion, sports related concussion (SRC), mild traumatic brain injury (mTBI), traumatic brain injury (TBI), and the like.

In one embodiment, the invention provides a biomarker for the detection of brain injury in a subject. In one embodiment, the biomarker for the detection of brain injury includes but is not limited to those listed in Table 4A, Table 4B, Table 5A, Table 5B, Table 6, Table 7A, Table 7B, Table 8A, Table 8B, Table 9A, and Table 9B.

In one embodiment, the invention provides a biomarker for the detection of recovery from brain injury in a subject. In one embodiment, the biomarker for the detection of recovery from brain injury includes but is not limited to those listed in Table 4A, Table 4B, Table 5A, Table 5B, Table 6, Table 7A, Table 7B, Table 8A, Table 8B, Table 9A, and Table 9B.

In one embodiment, the method comprises determining that the level of one or more biomarkers listed in Table 4A is upregulated in a biological sample obtained acutely after head trauma as compared to control. In one embodiment, the method comprises determining that the level of one or more biomarkers listed in Table 4B is downregulated in a biological sample obtained acutely after head trauma as compared to control. In one embodiment, the method comprises determining that the level of one or more biomarkers listed in Table 7A is upregulated in a biological sample obtained acutely after head trauma as compared to control. In one embodiment, the method comprises determining that the level of one or more biomarkers listed in Table 7B is downregulated in a biological sample obtained acutely after head trauma as compared to control. In one embodiment, the biological sample obtained acutely after head trauma comprises a sample obtained less than about 24 hours after head trauma. In one embodiment, the biological sample obtained acutely after head trauma is obtained about 6 hours after head trauma.

In one embodiment, the method comprises determining that the level of one or more biomarkers listed in Table 5A is upregulated in a biological sample obtained sub-acutely after head trauma as compared to control. In one embodiment, the method comprises determining that the level of one or more biomarkers listed in Table 5B is downregulated in a biological sample obtained sub-acutely after head trauma as compared to control. In one embodiment, the method comprises determining that the level of one or more biomarkers listed in Table 8A is upregulated in a biological sample obtained sub-acutely after head trauma as compared to control. In one embodiment, the method comprises determining that the level of one or more biomarkers listed in Table 8B is downregulated in a biological sample obtained sub-acutely after head trauma as compared to control. In one embodiment, the biological sample obtained sub-acutely after head trauma comprises a sample obtained about 1 day to about 14 days after head trauma. In one embodiment, the biological sample obtained sub-acutely after head trauma is obtained about 7 days after head trauma.

### Identifying a Marker or Biomarker

The invention includes methods for diagnosing brain injury, assessing the severity of brain injury, and assessing the recovery from brain injury by detecting differentially expressed biomarkers in a biological sample obtained from a subject who has experienced head trauma as compared to a control or reference sample.

The invention contemplates the detection of differentially expressed markers by nucleic acid microarray. The invention further contemplates using methods known to those skilled in the art to detect and to measure the level of differentially expressed marker expression products, such as RNA and protein, to measure the level of one or more differentially expressed marker expression products.

Methods of detecting or measuring gene expression may utilize methods that focus on cellular components (cellular examination), or methods that focus on examining extracellular components (fluid examination). Because gene expression involves the ordered production of a number of different molecules, a cellular or fluid examination may be used to detect or measure a variety of molecules including RNA, protein, and a number of molecules that may be modified as a result of the protein's function. Typical diagnostic methods focusing on nucleic acids include amplification techniques such as PCR and RT-PCR (including quantitative variants), and hybridization techniques such as in situ hybridization, microarrays, blots, and others. Typical diagnostic methods focusing on proteins include binding techniques such as ELISA, immunohistochemistry, microarray and functional techniques such as enzymatic assays.

The genes identified as being differentially expressed may be assessed in a variety of nucleic acid detection assays to detect or quantify the expression level of a gene or multiple genes in a given sample. For example, traditional Northern blotting, nuclease protection, RT-PCR, microarray, and differential display methods may be used for detecting gene expression levels. Methods for assaying for mRNA include Northern blots, slot blots, dot blots, and hybridization to an ordered array of oligonucleotides. Any method for specifically and quantitatively measuring a specific protein or mRNA or DNA product can be used. However, methods and assays are most efficiently designed with array or chip hybridization-based methods for detecting the expression of a large number of genes. Any hybridization assay format may be used, including solution-based and solid support-based assay formats.

The protein products of the genes identified herein can also be assayed to determine the amount of expression. Methods for assaying for a protein include Western blot, immunoprecipitation, and radioimmunoassay. The proteins analyzed may be localized intracellularly (most commonly an application of immunohistochemistry) or extracellularly (most commonly an application of immunoassays such as ELISA).

Biological samples may be of any biological tissue or fluid. Frequently the sample will be a “clinical sample” which is a sample derived from a patient. The biological sample may contain any biological material suitable for detecting the desired biomarkers, and may comprise cellular and/or non-cellular material obtained from the individual. A biological sample can be obtained by appropriate methods, such as, by way of examples, blood draw, fluid draw, or biopsy. Examples of such samples include but are not limited to blood, lymph, urine, gynecological fluids, biopsies, amniotic fluid and smears. Samples that are liquid in nature are referred to herein as “bodily fluids.” Body samples may be obtained from a patient by a variety of techniques including, for example, by scraping or swabbing an area or by using a needle to aspirate bodily fluids. Methods for collecting various body samples are well known in the art. Frequently, a sample will be a “clinical sample,” i.e., a sample derived from a patient. Such samples include, but are not limited to, bodily fluids which may or may not contain cells, e.g., blood (e.g., whole blood, serum or plasma), urine, saliva, tissue or fine needle biopsy samples, and archival samples with known diagnosis, treatment and/or outcome history. In certain embodiments, the biological sample comprises a blood cell. In one embodiment, the biological sample comprises a peripheral mononuclear blood cell (PMBC).

In certain embodiments, the biological sample is obtained from the subject following head trauma. For example, in certain embodiments, the biological sample is obtained about 1 minute, 5 minutes, 10 minutes 30 minutes, 1 hour, 2 hours, 4 hours, 6 hours, 12 hours, 18 hours, 24 hours, 2 days, 3 days, 4 days, 5 days, 6 days, 7 days, 10 days, 2 weeks, 3 weeks, 4 weeks, 1 month, 2 months, 3 months, 4 months, 5 months, 6 months, 9 months, 1 year, 2 years following head trauma. In certain embodiments, the sample is obtained more than 2 years following head trauma. In certain embodiments, the sample is obtained less than 1 minute following head trauma. In certain embodiments, a plurality of biological samples are obtained at one or more different time points.

Control group samples may either be from a normal subject or samples from subjects with a known brain injury. In certain embodiments, the control sample may be either from subjects who have recovered or have not recovered from a known brain injury. As described below, comparison of the expression patterns of the sample to be tested with those of the controls can be used to diagnose brain injury, assess the severity of brain injury, or assess the recovery from brain injury. In some instances, the control groups are only for the purposes of establishing initial cutoffs or thresholds for the assays of the invention. Therefore, in some instances, the systems and methods of the invention can diagnose brain injury, assess the severity of brain injury, or assess the recovery from brain injury without the need to compare with a control group.

### Methods of Diagnosis

The present invention provides methods for diagnosing brain injury, assessing brain injury severity, and assessing recovery from brain injury in a subject who has experienced a head trauma. The present invention includes methods for identifying subjects who have a brain injury, including those subjects who are asymptomatic or only exhibit non-specific indicators of brain injury by detection of the biomarkers disclosed herein. These biomarkers are also useful for monitoring subjects undergoing treatments and therapies for brain injury and/or brain injury-related conditions, and for selecting or modifying therapies and treatments that would be efficacious in subjects having a brain injury, wherein selection and use of such treatments and therapies. Such treatments may treat the injury by slowing or preventing brain injury-associated symptoms such as nausea, light sensitivity, headache, cognitive deficits, psychological deficits, behavioral issues, and the like.

The invention provides improved methods for the diagnosis and prognosis of brain injury. The diagnosis or prognosis of brain injury can be assessed by measuring one or more of the biomarkers described herein, and comparing the measured values to comparator values, reference values, or index values. Such a comparison can be undertaken with mathematical algorithms or formula in order to combine information from results of multiple individual biomarkers and other parameters into a single measurement or index. Subjects identified as having a brain injury can optionally be selected to receive treatment regimens, such as administration of therapeutic compounds to prevent, treat or delay the brain injury-related symptoms.

Identifying a subject as having a brain injury within a few hours after trauma allows for the selection and initiation of various therapeutic interventions or treatment regimens in order to delay, reduce or prevent brain injury-related symptoms as well as improve recovery. Monitoring the levels of at least one biomarker also allows for the course of treatment to be monitored. For example, a sample can be provided from a subject undergoing treatment regimens or therapeutic interventions. Such treatment regimens or therapeutic interventions can include reduction of intracranial pressure, surgery, cognitive therapy, occupational therapy, speech therapy, physiotherapy, administration of pharmaceuticals, and treatment with therapeutics or prophylactics used in subjects diagnosed or identified with a brain injury. Samples can be obtained from the subject at various time points before, during, or after treatment.

The biomarkers of the present invention can thus be used to generate a biomarker profile or signature of the subjects: (i) who do not have a brain injury, (ii) who have a brain injury, and/or (iii) who are recovering or have recovered from a brain injury. The biomarker profile of a subject can be compared to a predetermined or comparator biomarker profile or reference biomarker profile to diagnose brain injury, to monitor the progression or rate of progression of brain injury-related symptoms or pathology, and to monitor the effectiveness of brain injury treatments. Data concerning the biomarkers of the present invention can also be combined or correlated with other data or test results, such as, without limitation, measurements of clinical parameters or other algorithms for brain injury. Other data includes age, ethnicity, body mass index (BMI), neurological testing (e.g., Glasgow Coma Score, ImPACT, BESS), EEG recording data, neuroimaging results (e.g., CT scan, MRI, angiography), and the like. The data may also comprise subject information such as medical history and any relevant family history.

The present invention also provides methods for identifying agents for treating brain injury that are appropriate or otherwise customized for a specific subject. In this regard, a test sample from a subject, exposed to a therapeutic agent or a drug, can be taken and the level of one or more biomarkers can be determined. The level of one or more biomarkers can be compared to a sample derived from the subject before and after treatment, or can be compared to samples derived from one or more subjects who have shown improvements in risk factors as a result of such treatment or exposure.

In some embodiments, the methods described herein may utilize a biological sample (such as urine, saliva, blood, serum, amniotic fluid, or tears), for the detection of one or more markers of the invention in the sample. In one embodiment, the method comprises detection of one or more markers in a PMBC of the subject.

In one embodiment, the invention provides a biomarker for the diagnosis of a brain injury in a subject. In one embodiment, the biomarker for the diagnosis of brain injury, includes but is not limited to the biomarkers listed in Table 4A, Table 4B, Table 5A, Table 5B, Table 6, Table 7A, Table 7B, Table 8A, Table 8B, Table 9A, and Table 9B.

In one embodiment, the invention provides a biomarker for the determining that a subject has recovered from a brain injury. In one embodiment, the biomarker for determining that a subject has recovered from a brain injury, includes but is not limited to the biomarkers listed in Table 4A, Table 4B, Table 5A, Table 5B, Table 6, Table 7A, Table 7B, Table 8A, Table 8B, Table 9A, and Table 9B.

In one embodiment, the method comprises detecting that one or more biomarkers listed in Table 4A is upregulated in a sample obtained in the acute period after head trauma, as compared to a control sample. In one embodiment, the method comprises detecting that one or more biomarkers listed in Table 4B is downregulated in a sample obtained in the acute period after trauma, as compared to a control sample. In certain embodiments, the sample obtained in the acute period after head trauma is obtained at about 6 hours after head trauma. In one embodiment, the control sample is the level of the one or more biomarkers at baseline, as measured in a sample obtained prior to head trauma.

In one embodiment, the method comprises detecting that one or more biomarkers listed in Table 5A is upregulated in a sample obtained in the sub-acute period after head trauma, as compared to a control sample. In one embodiment, the method comprises detecting that one or more biomarkers listed in Table 5B is downregulated in a sample obtained in the sub-acute period after trauma, as compared to a control sample. In certain embodiments, the sample obtained in the sub-acute period after head trauma is obtained at about 7 days after head trauma. In one embodiment, the control sample is the level of the one or more biomarkers at baseline, as measured in a sample obtained prior to head trauma.

In one embodiment, the method comprises detecting that one or more biomarkers listed in Table 6 is differentially expressed in a sample obtained in the sub-acute period after head trauma, as compared the level in a sample obtained in the acute period after head trauma. In certain embodiments, the sample obtained in the sub-acute period after head trauma is obtained at about 7 days after head trauma. In certain embodiments, the sample obtained in the acute period after head trauma is obtained at about 6 hours after head trauma. In one embodiment, the control sample is the level of the one or more biomarkers at baseline, as measured in a sample obtained prior to head trauma.

In one embodiment, the method comprises detecting that one or more biomarkers listed in Table 7A is upregulated in the acute period after head trauma, as compared to a control. For example, in one embodiment, the method comprises detecting the upregulation by determining that the change in expression of the one or more biomarkers listed in Table 7A in the subject from the acute period compared to baseline is greater than the change in expression of the one or more biomarkers listed in Table 7A in a control subject or population that has not experienced head trauma. In one embodiment, the method comprises detecting that one or more biomarkers listed in Table 7B is downregulated in the acute period after head trauma, as compared to a control. For example, in one embodiment, the method comprises detecting the downregulation by determining that the change in expression of the one or more biomarkers listed in Table 7B in the subject from the acute period compared to baseline is less than the change in expression of the one or more biomarkers listed in Table 7B in a control subject or population that has not experienced head trauma.

In one embodiment, the method comprises detecting that one or more biomarkers listed in Table 8A is upregulated in the subacute period after head trauma, as compared to a control. For example, in one embodiment, the method comprises detecting the upregulation by determining that the change in expression of the one or more biomarkers listed in Table 8A in the subject from the subacute period compared to baseline is greater than the change in expression of the one or more biomarkers listed in Table 8A in a control subject or population that has not experienced head trauma. In one embodiment, the method comprises detecting that one or more biomarkers listed in Table 8B is downregulated in the subacute period after head trauma, as compared to a control. For example, in one embodiment, the method comprises detecting the downregulation by determining that the change in expression of the one or more biomarkers listed in Table 8B in the subject from the subacute period compared to baseline is less than the change in expression of the one or more biomarkers listed in Table 8B in a control subject or population that has not experienced head trauma.

In one embodiment, the method comprises detecting that one or more biomarkers listed in Table 9A is upregulated in the subacute period after head trauma, as compared to a control. For example, in one embodiment, the method comprises detecting the upregulation by determining that the change in expression of the one or more biomarkers listed in Table 9A in the subject from the subacute period compared to the acute period is greater than the change in expression of the one or more biomarkers listed in Table 9A in a control subject or population that has not experienced head trauma. In one embodiment, the method comprises detecting that one or more biomarkers listed in Table 9B is downregulated in the subacute period after head trauma, as compared to a control. For example, in one embodiment, the method comprises detecting the downregulation by determining that the change in expression of the one or more biomarkers listed in Table 9B in the subject from the subacute period compared to the acute period is less than the change in expression of the one or more biomarkers listed in Table 9B in a control subject or population that has not experienced head trauma.

In one embodiment, the method comprises detecting one or more markers in a biological sample of the subject. In various embodiments, the level of one or more of markers of the invention in the biological test sample of the subject is compared with the level of the biomarker in a comparator. Non-limiting examples of comparators include, but are not limited to, a negative control, a positive control, standard control, standard value, an expected normal background value of the subject, a historical normal background value of the subject, a reference standard, a reference level, an expected normal background value of a population that the subject is a member of, or a historical normal background value of a population that the subject is a member of. In one embodiment, the comparator is a level of the one or more biomarker in a sample obtained from the subject prior to head trauma. In one embodiment, the comparator is a level of the one or more biomarker in an earlier obtained sample from the subject after head trauma but before the collection of the test sample.

In another embodiment, the invention is a method of monitoring the progression of diabetes in a subject by assessing the level of one or more of the markers of the invention in a biological sample of the subject.

In various embodiments, the subject is a human subject, and may be of any race, sex and age.

Information obtained from the methods of the invention described herein can be used alone, or in combination with other information (e.g., disease status, disease history, vital signs, blood chemistry, neurological score, etc.) from the subject or from the biological sample obtained from the subject.

In various embodiments of the methods of the invention, the level of one or more markers of the invention is determined to be increased when the level of one or more of the markers of the invention is increased by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator.

In other various embodiments of the methods of the invention, the level of one or more markers of the invention is determined to be decreased when the level of one or more of the markers of the invention is decreased by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator.

In the methods of the invention, a biological sample from a subject is assessed for the level of one or more of the markers of the invention in the biological sample obtained from the patient. The level of one or more of the markers of the invention in the biological sample can be determined by assessing the amount of polypeptide of one or more of the biomarkers of the invention in the biological sample, the amount of mRNA of one or more of the biomarkers of the invention in the biological sample, the amount of enzymatic activity of one or more of the biomarkers of the invention in the biological sample, or a combination thereof.

### Detecting a Biomarker

In one embodiment, the invention includes detecting one or more mRNA biomarkers, polypeptide biomarkers, or a combination thereof in a biological sample. Biomarkers generally can be measured and detected through a variety of assays, methods and detection systems known to one of skill in the art.

Various methods include but are not limited to immunoassays, microarray, PCR, RT-PCR, refractive index spectroscopy (RI), ultra-violet spectroscopy (UV), fluorescence analysis, electrochemical analysis, radiochemical analysis, near-infrared spectroscopy (near-IR), infrared (IR) spectroscopy, nuclear magnetic resonance spectroscopy (NMR), light scattering analysis (LS), mass spectrometry, pyrolysis mass spectrometry, nephelometry, dispersive Raman spectroscopy, gas chromatography, liquid chromatography, gas chromatography combined with mass spectrometry, liquid chromatography combined with mass spectrometry, matrix-assisted laser desorption ionization-time of flight (MALDI-TOF) combined with mass spectrometry, ion spray spectroscopy combined with mass spectrometry, capillary electrophoresis, colorimetry and surface plasmon resonance (such as according to systems provided by Biacore Life Sciences). See also PCT Publications WO/2004/056456 and WO/2004/088309. In this regard, biomarkers can be measured using the above-mentioned detection methods, or other methods known to the skilled artisan. Other biomarkers can be similarly detected using reagents that are specifically designed or tailored to detect them.

Different types of biomarkers and their measurements can be combined in the compositions and methods of the present invention. In various embodiments, the protein form of the biomarkers is measured. In various embodiments, the nucleic acid form of the biomarkers is measured. In exemplary embodiments, the nucleic acid form is mRNA. In various embodiments, measurements of protein biomarkers are used in conjunction with measurements of nucleic acid biomarkers.

In various embodiments of the invention, methods of measuring polypeptide levels in a biological sample obtained from a subject include, but are not limited to, an immunochromatography assay, an immunodot assay, a Luminex assay, an ELISA assay, an ELISPOT assay, a protein microarray assay, a ligand-receptor binding assay, displacement of a ligand from a receptor assay, displacement of a ligand from a shared receptor assay, an immunostaining assay, a Western blot assay, a mass spectrophotometry assay, a radioimmunoassay (RIA), a radioimmunodiffusion assay, a liquid chromatography-tandem mass spectrometry assay, an ouchterlony immunodiffusion assay, reverse phase protein microarray, a rocket immunoelectrophoresis assay, an immunohistostaining assay, an immunoprecipitation assay, a complement fixation assay, FACS, an enzyme-substrate binding assay, an enzymatic assay, an enzymatic assay employing a detectable molecule, such as a chromophore, fluorophore, or radioactive substrate, a substrate binding assay employing such a substrate, a substrate displacement assay employing such a substrate, and a protein chip assay (see also, 2007, Van Emon, Immunoassay and Other Bioanalytical Techniques, CRC Press; 2005, Wild, Immunoassay Handbook, Gulf Professional Publishing; 1996, Diamandis and Christopoulos, Immunoassay, Academic Press; 2005, Joos, Microarrays in Clinical Diagnosis, Humana Press; 2005, Hamdan and Righetti, Proteomics Today, John Wiley and Sons; 2007).

Methods for detecting a nucleic acid (e.g., mRNA), such as RT-PCR, real time PCR, microarray, branch DNA, NASBA and others, are well known in the art. Using sequence information provided by the database entries for the biomarker sequences, expression of the biomarker sequences can be detected (if present) and measured using techniques well known to one of ordinary skill in the art. For example, sequences in sequence database entries or sequences disclosed herein can be used to construct probes for detecting biomarker RNA sequences in, e.g., Northern blot hybridization analyses or methods which specifically, and, preferably, quantitatively amplify specific nucleic acid sequences. As another example, the sequences can be used to construct primers for specifically amplifying the biomarker sequences in, e.g., amplification-based detection methods such as reverse-transcription based polymerase chain reaction (RT-PCR). When alterations in gene expression are associated with gene amplification, deletion, polymorphisms and mutations, sequence comparisons in test and reference populations can be made by comparing relative amounts of the examined DNA sequences in the test and reference cell populations. In addition to Northern blot and RT-PCR, RNA can also be measured using, for example, other target amplification methods (e.g., TMA, SDA, NASBA), signal amplification methods (e.g., bDNA), nuclease protection assays, in situ hybridization and the like.

In some embodiments, quantitative hybridization methods, such as Southern analysis, Northern analysis, or in situ hybridizations, can be used (see Current Protocols in Molecular Biology, Ausubel, F. et al., eds., John Wiley & Sons, including all supplements). A “nucleic acid probe,” as used herein, can be a DNA probe or an RNA probe. The probe can be, for example, a gene, a gene fragment (e.g., one or more exons), a vector comprising the gene, a probe or primer, etc. For representative examples of use of nucleic acid probes, see, for example, U.S. Pat. Nos. 5,288,611 and 4,851,330. The nucleic acid probe can be, for example, a full-length nucleic acid molecule, or a portion thereof, such as an oligonucleotide of at least 15, 30, 50, 100, 250 or 500 nucleotides in length and sufficient to specifically hybridize under stringent conditions to appropriate target mRNA or cDNA. The hybridization sample is maintained under conditions which are sufficient to allow specific hybridization of the nucleic acid probe to mRNA or cDNA. Specific hybridization can be performed under high stringency conditions or moderate stringency conditions, as appropriate. In a preferred embodiment, the hybridization conditions for specific hybridization are high stringency. Specific hybridization, if present, is then detected using standard methods. If specific hybridization occurs between the nucleic acid probe having a mRNA or cDNA in the test sample, the level of the mRNA or cDNA in the sample can be assessed. More than one nucleic acid probe can also be used concurrently in this method. Specific hybridization of any one of the nucleic acid probes is indicative of the presence of the mRNA or cDNA of interest, as described herein.

Alternatively, a peptide nucleic acid (PNA) probe can be used instead of a nucleic acid probe in the quantitative hybridization methods described herein. PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, 1994, Nielsen et al., Bioconjugate Chemistry 5:1). The PNA probe can be designed to specifically hybridize to a target nucleic acid sequence. Hybridization of the PNA probe to a nucleic acid sequence is used to determine the level of the target nucleic acid in the biological sample.

In another embodiment, arrays of oligonucleotide probes that are complementary to target nucleic acid sequences in the biological sample obtained from a subject can be used to determine the level of one or more biomarkers in the biological sample obtained from a subject. The array of oligonucleotide probes can be used to determine the level of one or more biomarkers alone, or the level of the one or more biomarkers in relation to the level of one or more other nucleic acids in the biological sample. Oligonucleotide arrays typically comprise a plurality of different oligonucleotide probes that are coupled to a surface of a substrate in different known locations. These oligonucleotide arrays, also known as “Genechips,” have been generally described in the art, for example, U.S. Pat. No. 5,143,854 and PCT patent publication Nos. WO 90/15070 and 92/10092. These arrays can generally be produced using mechanical synthesis methods or light directed synthesis methods which incorporate a combination of photolithographic methods and solid phase oligonucleotide synthesis methods. See Fodor et al., Science, 251:767-777 (1991), Pirrung et al., U.S. Pat. No. 5,143,854 (see also PCT Application No. WO 90/15070) and Fodor et al., PCT Publication No. WO 92/10092 and U.S. Pat. No. 5,424,186. Techniques for the synthesis of these arrays using mechanical synthesis methods are described in, e.g., U.S. Pat. No. 5,384,261.

After an oligonucleotide array is prepared, a nucleic acid of interest is hybridized with the array and its level is quantified. Hybridization and quantification are generally carried out by methods described herein and also in, e.g., published PCT Application Nos. WO 92/10092 and WO 95/11995, and U.S. Pat. No. 5,424,186. In brief, a target nucleic acid sequence is amplified by well-known amplification techniques, e.g., PCR. Typically, this involves the use of primer sequences that are complementary to the target nucleic acid. Asymmetric PCR techniques may also be used. Amplified target, generally incorporating a label, is then hybridized with the array under appropriate conditions. Upon completion of hybridization and washing of the array, the array is scanned to determine the quantity of hybridized nucleic acid. The hybridization data obtained from the scan is typically in the form of fluorescence intensities as a function of quantity, or relative quantity, of the target nucleic acid in the biological sample. The target nucleic acid can be hybridized to the array in combination with one or more comparator controls (e.g., positive control, negative control, quantity control, etc.) to improve quantification of the target nucleic acid in the sample.

The probes and primers according to the invention can be labeled directly or indirectly with a radioactive or nonradioactive compound, by methods well known to those skilled in the art, in order to obtain a detectable and/or quantifiable signal; the labeling of the primers or of the probes according to the invention is carried out with radioactive elements or with nonradioactive molecules. Among the radioactive isotopes used, mention may be made of ³²P, ³³P, ³⁵S or ³H. The nonradioactive entities are selected from ligands such as biotin, avidin, streptavidin or digoxigenin, haptenes, dyes, and luminescent agents such as radioluminescent, chemiluminescent, bioluminescent, fluorescent or phosphorescent agents.

Nucleic acids can be obtained from the cells using known techniques. Nucleic acid herein refers to RNA, including mRNA, and DNA, including cDNA. The nucleic acid can be double-stranded or single-stranded (i.e., a sense or an antisense single strand) and can be complementary to a nucleic acid encoding a polypeptide. The nucleic acid content may also be an RNA or DNA extraction performed on a biological sample, including a biological fluid and fresh or fixed tissue sample.

There are many methods known in the art for the detection and quantification of specific nucleic acid sequences and new methods are continually reported. A great majority of the known specific nucleic acid detection and quantification methods utilize nucleic acid probes in specific hybridization reactions. Preferably, the detection of hybridization to the duplex form is a Southern blot technique. In the Southern blot technique, a nucleic acid sample is separated in an agarose gel based on size (molecular weight) and affixed to a membrane, denatured, and exposed to (admixed with) the labeled nucleic acid probe under hybridizing conditions. If the labeled nucleic acid probe forms a hybrid with the nucleic acid on the blot, the label is bound to the membrane.

In the Southern blot, the nucleic acid probe is preferably labeled with a tag. That tag can be a radioactive isotope, a fluorescent dye or the other well-known materials. Another type of process for the specific detection of nucleic acids in a biological sample known in the art are the hybridization methods as exemplified by U.S. Pat. No. 6,159,693 and U.S. Pat. No. 6,270,974, and related patents. To briefly summarize one of those methods, a nucleic acid probe of at least 10 nucleotides, preferably at least 15 nucleotides, more preferably at least 25 nucleotides, having a sequence complementary to a nucleic acid of interest is hybridized in a sample, subjected to depolymerizing conditions, and the sample is treated with an ATP/luciferase system, which will luminesce if the nucleic sequence is present. In quantitative Southern blotting, the level of the nucleic acid of interest can be compared with the level of a second nucleic acid of interest, and/or to one or more comparator control nucleic acids (e.g., positive control, negative control, quantity control, etc.).

Many methods useful for the detection and quantification of nucleic acid takes advantage of the polymerase chain reaction (PCR). The PCR process is well known in the art (U.S. Pat. No. 4,683,195, No. 4,683,202, and No. 4,800,159). To briefly summarize PCR, nucleic acid primers, complementary to opposite strands of a nucleic acid amplification target sequence, are permitted to anneal to the denatured sample. A DNA polymerase (typically heat stable) extends the DNA duplex from the hybridized primer. The process is repeated to amplify the nucleic acid target. If the nucleic acid primers do not hybridize to the sample, then there is no corresponding amplified PCR product. In this case, the PCR primer acts as a hybridization probe.

In PCR, the nucleic acid probe can be labeled with a tag as discussed elsewhere herein. Most preferably the detection of the duplex is done using at least one primer directed to the nucleic acid of interest. In yet another embodiment of PCR, the detection of the hybridized duplex comprises electrophoretic gel separation followed by dye-based visualization.

Typical hybridization and washing stringency conditions depend in part on the size (i.e., number of nucleotides in length) of the oligonucleotide probe, the base composition and monovalent and divalent cation concentrations (Ausubel et al., 1994, eds Current Protocols in Molecular Biology).

In a preferred embodiment, the process for determining the quantitative and qualitative profile of the nucleic acid of interest according to the present invention is characterized in that the amplifications are real-time amplifications performed using a labeled probe, preferably a labeled hydrolysis-probe, capable of specifically hybridizing in stringent conditions with a segment of the nucleic acid of interest. The labeled probe is capable of emitting a detectable signal every time each amplification cycle occurs, allowing the signal obtained for each cycle to be measured.

The real-time amplification, such as real-time PCR, is well known in the art, and the various known techniques will be employed in the best way for the implementation of the present process. These techniques are performed using various categories of probes, such as hydrolysis probes, hybridization adjacent probes, or molecular beacons. The techniques employing hydrolysis probes or molecular beacons are based on the use of a fluorescence quencher/reporter system, and the hybridization adjacent probes are based on the use of fluorescence acceptor/donor molecules.

Hydrolysis probes with a fluorescence quencher/reporter system are available in the market, and are for example commercialized by the Applied Biosystems group (USA). Many fluorescent dyes may be employed, such as FAM dyes (6-carboxy-fluorescein), or any other dye phosphoramidite reagents.

Among the stringent conditions applied for any one of the hydrolysis-probes of the present invention is the Tm, which is in the range of about 65° C. to 75° C. Preferably, the Tm for any one of the hydrolysis-probes of the present invention is in the range of about 67° C. to about 70° C. Most preferably, the Tm applied for any one of the hydrolysis-probes of the present invention is about 67° C.

In one aspect, the invention includes a primer that is complementary to a nucleic acid of interest, and more particularly the primer includes 12 or more contiguous nucleotides substantially complementary to the nucleic acid of interest. Preferably, a primer featured in the invention includes a nucleotide sequence sufficiently complementary to hybridize to a nucleic acid sequence of about 12 to 25 nucleotides.

More preferably, the primer differs by no more than 1, 2, or 3 nucleotides from the target flanking nucleotide sequence In another aspect, the length of the primer can vary in length, preferably about 15 to 28 nucleotides in length (e.g., 16, 17, 18, 19, 20, 21, 22, 23, 24, 25, 26, or 27 nucleotides in length).

The concentration of the biomarker in a sample may be determined by any suitable assay. A suitable assay may include one or more of the following methods, an enzyme assay, an immunoassay, mass spectrometry, chromatography, electrophoresis or an antibody microarray, or any combination thereof. Thus, as would be understood by one skilled in the art, the system and methods of the invention may include any method known in the art to detect a biomarker in a sample.

The invention described herein also relates to methods for a multiplex analysis platform. In one embodiment, the method comprises an analytical method for multiplexing analytical measurements of markers.

### Kits

The present invention also pertains to kits useful in the methods of the invention. Such kits comprise various combinations of components useful in any of the methods described elsewhere herein, including for example, materials for quantitatively analyzing a biomarker of the invention (e.g., polypeptide and/or nucleic acid), materials for assessing the activity of a biomarker of the invention (e.g., polypeptide and/or nucleic acid), and instructional material. For example, in one embodiment, the kit comprises components useful for the quantification of a desired nucleic acid in a biological sample. In another embodiment, the kit comprises components useful for the quantification of a desired polypeptide in a biological sample. In a further embodiment, the kit comprises components useful for the assessment of the activity (e.g., enzymatic activity, substrate binding activity, etc.) of a desired polypeptide in a biological sample.

In a further embodiment, the kit comprises the components of an assay for monitoring the effectiveness of a treatment administered to a subject in need thereof, containing instructional material and the components for determining whether the level of a biomarker of the invention in a biological sample obtained from the subject is modulated during or after administration of the treatment. In various embodiments, to determine whether the level of a biomarker of the invention is modulated in a biological sample obtained from the subject, the level of the biomarker is compared with the level of at least one comparator control contained in the kit, such as a positive control, a negative control, a historical control, a historical norm, or the level of another reference molecule in the biological sample. In certain embodiments, the ratio of the biomarker and a reference molecule is determined to aid in the monitoring of the treatment.

### Treatments

In certain embodiments, treatment comprises administering a disease-modulating drug to a subject. The drug can be a therapeutic or prophylactic used in subjects diagnosed or identified with a disease or at risk of having the disease. In certain embodiments, modifying therapy refers to altering the duration, frequency or intensity of therapy, for example, altering dosage levels.

In various embodiments, effecting a therapy comprises causing a subject to or communicating to a subject the need to undergoing rehabilitation therapy, for example speech therapy, occupational therapy, physiotherapy, etc. The therapy can also include surgery.

Measurement of biomarker levels allow for the course of treatment of a disease to be monitored. The effectiveness of a treatment regimen for a disease can be monitored by detecting one or more biomarkers in an effective amount from samples obtained from a subject over time and comparing the amount of biomarkers detected. For example, a first sample can be obtained prior to the subject receiving treatment and one or more subsequent samples are taken after or during treatment of the subject. Changes in biomarker levels across the samples may provide an indication as to the effectiveness of the therapy.

To identify therapeutics or drugs that are appropriate for a specific subject, a test sample from the subject can also be exposed to a therapeutic agent or a drug, and the level of one or more biomarkers can be determined. Biomarker levels can be compared to a sample derived from the subject before and after treatment or exposure to a therapeutic agent or a drug, or can be compared to samples derived from one or more subjects who have shown improvements relative to a disease as a result of such treatment or exposure. Thus, in one aspect, the invention provides a method of assessing the efficacy of a therapy with respect to a subject comprising taking a first measurement of a biomarker panel in a first sample from the subject; effecting the therapy with respect to the subject; taking a second measurement of the biomarker panel in a second sample from the subject and comparing the first and second measurements to assess the efficacy of the therapy.

Additionally, therapeutic agents suitable for administration to a particular subject can be identified by detecting one or more biomarkers in an effective amount from a sample obtained from a subject and exposing the subject-derived sample to a test compound that determines the amount of the biomarker(s) in the subject-derived sample. Accordingly, treatments or therapeutic regimens for use in subjects having a brain injury can be selected based on the amounts of biomarkers in samples obtained from the subjects and compared to a reference value. Two or more treatments or therapeutic regimens can be evaluated in parallel to determine which treatment or therapeutic regimen would be the most efficacious for use in a subject to delay onset, or slow progression of a disease. In various embodiments, a recommendation is made on whether to initiate or continue treatment of a disease.

In various exemplary embodiments, effecting a therapy comprises administering a disease-modulating drug to the subject. The subject may be treated with one or more drugs until altered levels of the measured biomarkers return to a baseline value measured in a population not having an injury, having a less severe injury, or showing improvements in disease biomarkers as a result of treatment with a drug. In one embodiment, the subject may be treated with one or more drugs until altered levels of the measured biomarkers return to a baseline value measured in pre-head trauma sample obtained from the subject. Additionally, improvements related to a changed level of a biomarker or clinical parameter may be the result of treatment with a disease-modulating drug.

Any drug or combination of drugs disclosed herein may be administered to a subject to treat a disease. The drugs herein can be formulated in any number of ways, often according to various known formulations in the art or as disclosed or referenced herein.

In various embodiments, any drug or combination of drugs disclosed herein is not administered to a subject to treat a disease. In these embodiments, the practitioner may refrain from administering the drug or combination of drugs, may recommend that the subject not be administered the drug or combination of drugs or may prevent the subject from being administered the drug or combination of drugs.

In various embodiments, one or more additional drugs may be optionally administered in addition to those that are recommended or have been administered. An additional drug will typically not be any drug that is not recommended or that should be avoided.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1: Genome-Wide Changes in Gene Expression Following Sports-Related Concussion

Despite up to 3.8 million Americans suffering a sports-related concussion (SRC) each year, no approved treatments exist. It is likely that this gap results from a limited understanding of the key post-SRC cellular changes. Peripheral transcriptome analysis can be used to estimate gene expression changes in the brain, providing clues into the physiologic underpinnings of recovery from SRC.

The experiments described herein were conducted to prospectively determine changes in global gene expression following SRC. Between 2010 and 2012, 253 contact athletes, underwent collection of peripheral blood mononuclear cells (PBMC) at the start of the sport season (baseline). Sixteen athletes who subsequently developed a SRC, along with 16 non-concussed teammates who served as controls, underwent repeat collection of PBMC within 6 hours of injury (acutely). Concussed athletes underwent additional PBMC collection at 7 days post-injury (sub-acutely).

Using Affymetrix microarray assays, PBMC mRNA expression at baseline was compared to mRNA expression acutely and sub-acutely post-SRC. PBMC samples from uninjured teammate control athletes were used to estimate the contribution of physical exertion to post-SRC gene changes. Ingenuity Pathway Analysis was used to translate differential gene expression into gene networks most likely affected by SRC. Clinical recovery was determined by examining changes in post-concussive symptoms, postural stability, and cognition from baseline to the sub-acute time point.

It is reported herein that athletes with SRC had significant changes in mRNA expression at both the acute and sub-acute time points compared to their baseline profiles. There were no significant gene expression changes among uninjured teammate control athletes. Acute transcriptional changes were centered on inflammatory activity with key transcriptional hubs being interleukins 6 and 12, toll-like receptor 4, and NF-κB. Sub-acute gene expression changes were centered on glucocorticoid receptor signaling with NF-κB, follicle stimulating hormone, chorionic gonadotropin, and protein kinase catalytic subunit being the key transcriptional hubs. No significant changes were observed in sub-acute post-concussive symptoms, postural stability, or cognition and all concussed athletes were recovered by the sub-acute time point.

Acute post-SRC gene transcriptional changes reflect regulation of the innate immune response as well as the transition to an acquired, adaptive immune response. By 7 days post-injury, transcriptional activity is centered on the regulation of the hypothalamic-pituitary-adrenal axis. These findings illustrate a time-dependent shift in gene expression post-injury that may provide insight into the pathophysiology of recovery from SRC.

The materials and methods employed in these experiments are now described.

Patients

Between 2010 and 2012, pre-season (baseline) PBMC samples were collected from 253 National Collegiate Athletic Association Division III contact sport athletes, and then banked. These athletes were followed prospectively for the development of a SRC, which was defined as an injury witnessed by an on-field coach or certified athletic trainer and meeting the definition of concussion as defined by the Sport Concussion Assessment Tool 2 (McCrory et al., 2009, Journal of Athletic Training, 44: 434-448). In brief, this tool provides a structured framework for evaluating 22 post concussive symptoms as well as orientation, memory, recall, balance and gait. Deficiencies in any of these areas were used to confirm suspicion of concussion. In athletes with a SRC, a second PBMC sample was obtained within 6 hours of injury (acute sample) and a third PBMC sample was obtained at 7 days post-injury (sub-acute sample).

Because physical exertion alone can potentially produce changes in gene expression (Connolly et al., 2004, Journal of Applied Physiology, 97:1461-1469), a comparison of gene changes before and after SRC potentially identifies genes not only related to SRC, but also to physical exertion. In order to estimate the contribution of physical exertion to post-SRC gene changes, a non-injured control group was examined. Non-injured athlete controls were identified at the time of each SRC. Athletes who supplied a baseline pre-season PBMC sample were eligible to serve as controls when one of their teammates suffered a SRC. Controls were matched to the concussed athlete for gender, team, and sport. A further eligibility requirement for controls was that they must have provided the baseline preseason PBMC sample in the same month and year as the concussed athlete, thus controlling for the time interval between baseline and injury. Both the concussed athlete and the non-injured teammate control athlete underwent phlebotomy for PBMC sampling at the same time acutely (i.e. within 6 hours) post injury. The concussed athlete, but not the uninjured teammate control, underwent repeat phlebotomy at the sub-acute time point. This is because it was assumed that the mRNA expression among uninjured control athletes would not change significantly over the 6 days spanning the acute-to-sub-acute time period. Unlike the injured athletes, who ceased all physical exertion after concussion, there was no change in the exertional activities of controls between the acute and subacute time points.

Clinical Outcome after SRC

Clinical outcome after SRC was determined by changes in cognitive performance, post-concussive symptoms, and postural stability, according to the recommendations of the 3rd International Conference on Concussion in Sport (McCrory et al., 2012, J Athl Train, 48: 554-575). All participating athletes underwent baseline, pre-season determination of cognition and postural stability with Immediate Post-Concussion Assessment and Cognitive Testing (ImPACT) and the Balance Error Scoring System (BESS), respectively. ImPACT and BESS testing were repeated in all subjects 7 days post-injury. ImPACT is a proprietary computer program that measures verbal memory, visual memory, reaction time, and visuomotor speed (Collins et al., 2003, Clinical Journal of Sport Medicine 13:222-229). ImPACT also includes a post-concussive symptom inventory. Normal day-to-day variation (termed “reliable change”) has been determined for each of these cognitive domains: verbal memory 8.75 points, visual memory 13.55 points, visuomotor speed 4.98 points, post-concussion symptom score 9.18 points, and reaction time 0.06 seconds (Iverson et al., 2003, Clin Neuropsychol, 13: 460-467). A significant change in a specific cognitive domain was thus defined as change exceeding the reliable change for that domain. BESS requires the athlete to stand in 3 different stances (double leg, single leg, and in tandem) for 20 seconds with eyes closed. Each stance is performed on a firm surface and on a 10-cm thick foam pad. The BESS score is calculated by adding 1 error point for each performance error, with a maximum of 10 errors per stance (Guskiewicz, 2001, Clin J Sport Med, 11: 182-189. Unlike ImPACT, there are no accepted reliable change values for BESS.

PBMC and RNA Isolation

PBMCs were isolated within 1 hour of venous blood collection following the protocol described in detail elsewhere (Kanof et al., 2001, Current Protocols in Immunology, Chapter 7: Unit 7.1). Isolated PBMC pellets were suspended in complete RPMI-10 medium and moved to storage in a −80 degrees centigrade freezer, after which they were stored at −190 degrees centigrade until analysis. Total RNA was isolated from PBMCs using TRIzol® Plus RNA Purification Kits (Life Technologies; Grand Island, N.Y.), and was treated with DNase I-Amplification Grade Kits (Life Technologies; Grand Island, N.Y.). The purity and concentration of RNA samples were verified using a NanoDrop DN-1000 spectrophotometer (Thermo Fisher Scientific; Wilmington, Del.). RNA integrity was determined on an Agilent bioanalyzer 2100 using the RNA 6000 Nano kit (Agilent; Santa Clara, Calif.). The quality of mRNA was evaluated in each sample. Samples with RNA Integrity Number (RIN) less than 7.0 were excluded from analysis.

Microarray

Using the GeneChip 3′ IVT Expression kit, each RNA (100 ng) sample was reverse transcribed, converted to biotinylated cRNA, and hybridized to Affymetrix HG-U133 Plus 2.0 microarrays (Affymetrix Inc; Santa Clara, Calif.), which contain 54,675 probesets representing 38,500 specific human genes. After staining with streptavidin-phycoerythrin and thorough washing, the raw data were obtained by laser scanning imaging.

Data Analysis

Demographic variables were compared in concussed athletes and uninjured teammate controls using Students t-test for age, and Fishers exact test for gender, race and sport. Recovery in cognitive function was determined by the percentage of athletes in each of the five cognitive domains displaying changes not exceeding reliable change. In addition, the mean score for each domain preinjury was compared to the score post injury using a paired t-test. Recovery in postural was determined by comparing the mean score for each stance preinjury to the score post injury using a paired t-test. Statistical significance was defined as a p-value less than or equal to 0.05.

Partek Genomics Suite software, version 6.6 (Partek Inc; St. Louis, Mo.), was utilized for all analyses performed on microarray data. Interrogating probes were imported, and corrections for background signal were applied using the robust multi-array average method, with additional corrections applied for the GC-content of probes. The probesets were standardized using quantile normalization, and expression levels of each probe underwent log-2 transformation to normalize data distributions. Parameters for identifying differentially expressed genes over time (i.e., within subject comparison) were then identified using analyses of variance of each probe set's expression level as a function of time point (baseline, acute, or sub-acute). Restricted maximum likelihood method was employed to fit the fixed and random effects of the design separately. In order to estimate the contribution of physical exertion to post-SRC gene changes, the same pre-post gene expression comparisons were planned among uninjured teammate controls athletes. Significant gene expression changes were defined as those increasing or decreasing by at least 1.5-fold (from baseline) and a p-value threshold with a false discovery rate (FDR)<0.05 corrected for multiple comparisons.

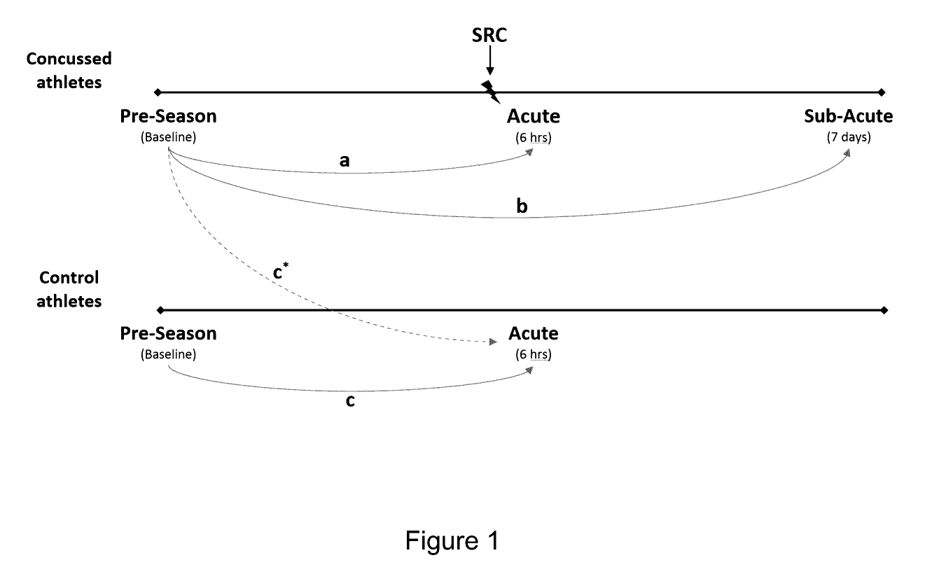

A Partek-generated heat map was used to display differential gene expression at baseline as well as acutely and sub-acutely post-SRC. Significant changes in differentially expressed genes were identified by comparing gene expression at baseline to expression acutely after SRC, and by comparing gene expression at baseline to expression sub-acutely after SRC (FIG. 1).

The functional biologic networks associated with these significantly changed genes were identified using Ingenuity Pathway Analysis (IPA) (Qiagen Ingenuity Systems Inc; Redwood City, Calif.). Central transcriptional nodes (“hubs”) were identified from the IPA-generated networks. Within each network, genes were ranked by the number of direct and indirect connections made with other genes. The top four genes with the most connections were considered transcriptional hubs (Barabasi et al., 2011, Nature Reviews Genetics, 12: 56-68)

The results of the experiments are now described.

Subjects and mRNA Samples

Of the 253 athletes enrolled, 16 (6%) suffered a concussion during the study period. Sixteen uninjured teammate controls were enrolled during the same time period. Compared to control athletes, concussed athletes were older and more likely to be Caucasian (Table 1).

TABLE 1
Demographics of concussed (n = 16) and control (n =
16) athletes.

| | Athletes | | Controls | | |
|---|---|---|---|---|---|
| | n | (%) | n | (%) | p-value |
| Age (mean, SD) | 19.38 | (1.47) | 18.53 | (0.41) | 0.035 |
| Gender | | | | | 0.784 |
| Female | 8 | (50%) | 9 | (56%) | |
| Male | 8 | (50%) | 7 | (44%) | |
| Race | | | | | 0.018 |
| White | 16 | (100%) | 10 | (62.5%) | |
| Not Reported | 0 | (0%) | 6 | (37.5%) | |
| Sport | | | | | 0.093 |
| Football | 6 | (38%) | 7 | (44%) | |
| Hockey | 4 | (25%) | 0 | (0%) | |
| Lacrosse | 1 | (6%) | 0 | (0%) | |
| Soccer | 5 | (31%) | 9 | (56%) | |

Of the samples obtained from the 16 concussed subjects, 15 had adequate mRNA (RIN >7) at baseline and the sub-acute time point, while 9 had adequate RNA at the acute time point. Of the samples obtained from the 16 uninjured athlete controls, none had adequate mRNA at baseline but all 16 had adequate RNA integrity at the acute time period. Because baseline samples from uninjured control athletes were unsuitable for analysis, baseline samples from athletes who subsequently suffered a concussion (i.e., before they were injured) were used as a surrogate for control baseline mRNA expression (FIG. 1).

### Clinical Outcomes

Among concussed athletes, there was no significant difference in mean cognitive performance on ImPACT pre-injury compared to the sub-acute time point (Table 2). No concussed athlete had changes exceeding reliable change in any of the five cognitive domains measured. Similarly, there was no significant difference in mean postural stability on BESS pre-injury compared to sub-acutely post-injury (Table 3).

TABLE 2
Mean (SD) ImPACT performance among concussed athletes (n =

| | Baseline | Day 7 | p-value |
|---|---|---|---|
| Verbal Memory Score | 88.13 (8.52) | 91.43 (6.88) | 0.211 |
| Visual Memory Score | 75.75 (15.81) | 75.79 (12.95) | 0.838 |
| Visual Motor Speed Score | 41.82 (5.90) | 43.91 (5.64) | 0.118 |
| Reaction Time (s) | 0.58 (0.07) | 0.54 (0.06) | 0.051 |
| Impulse Control Score | 5.13 (3.24) | 5.79 (2.94) | 0.459 |
| Total Symptom Score | 3.25 (7.49) | 2.57 (4.07) | 0.842 |
| Cognitive Efficiency Index | 0.39 (0.12) | 0.46 (0.08) | 0.059 |

TABLE 3
Mean (SD) errors during BESS assessment of
concussed athletes (n = 16).

| | Baseline | Day 7 | p-value |
|---|---|---|---|
| DL Floor | 0 (0.00) | 0.06 (0.25) | 0.334 |
| SL Floor | 3.31 (2.44) | 3.20 (1.86) | 0.506 |
| Tandom Floor | 1.06 (1.12) | 1.20 (1.21) | 0.583 |
| DL Foam | 0.13 (0.34) | 0.20 (0.41) | 0.671 |
| SL Foam | 7.81 (2.37) | 7.00 (1.77) | 0.222 |
| Tandom Foam | 4.56 (2.10) | 4.80 (2.88) | 0.628 |
| Total Errors | 16.88 (5.38) | 16.47 (5.26) | 0.819 |

Significant Changes in Gene Expression Before and after SRC

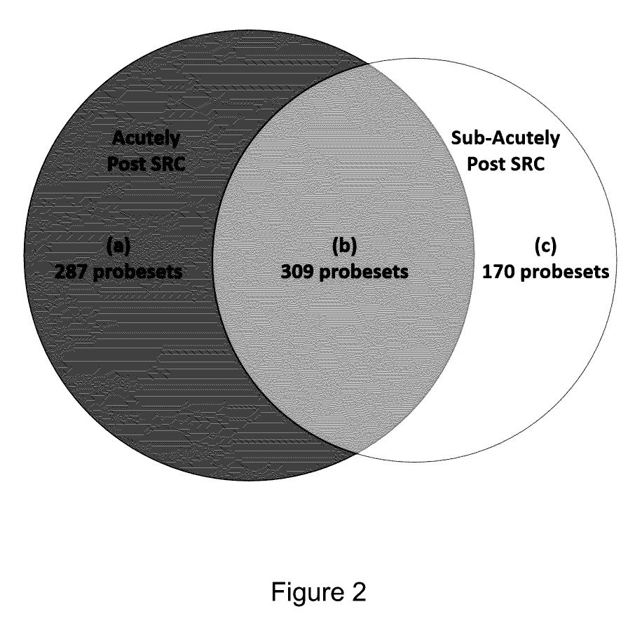

Of the 54,675 total probesets, 766 were found to have significant changes in expression level from baseline to post SRC among concussed athletes. The expression of 596 probesets was significantly changed from baseline to the acute timepoint, of which 287 were unique to this timepoint. The expression of 479 probesets was significantly changed from the baseline to sub-acute timepoint, of which 170 were unique to this timepoint. The expression of 309 probesets was significantly changed from baseline to both the acute and sub-acute timepoints (FIG. 2).

Twenty-five genes had >2 fold change in mRNA expression between baseline and the acute post-SRC timepoint (Table 4A and Table 4B). The genes with the largest decrease in expression from baseline to the acute post-SRC time point were chemokine (C-C motif) ligand 4 (CCL4) (3.4 fold) and RAR-related orphan receptor A (RORA) (2.7 fold). The genes with the largest increase in expression from baseline to the acute post-SRC time point were pyruvate dehydrogenase kinase, isozyme 4 (PDK4) (3.1 fold), and vacuole membrane protein 1 (VMP1) (2.6 fold).

Thirty-three genes had >2 fold change in mRNA expression between baseline and the subacute post-SRC timepoint (Table 5A and Table 5B). The genes with the largest decrease in expression from baseline to the sub-acute post-SRC time point were G0/G1switch 2 (G0S2) (7.7 fold), CCL3 (6.0 fold), and jun proto-oncogene (JUN) (4.7 fold). The genes with the largest increase in expression from baseline to the sub-acute post-SRC time point were EPM2A (laforin) interacting protein 1 (EPM2AIP1) (2.1 fold), and chemokine (C-X3-C motif) receptor 1 (CX3CR1) (1.9 fold).

Among uninjured control athletes, there were no significant changes in gene expression from baseline to the acute post-SRC time point.

A list of gene transcripts that are upregulated at 6 hours post SRC as compared to baseline is shown in Table 4A. A list of gene transcripts that are downregulated at 6 hours post SRC as compared to baseline is shown in Table 4B.

A list of gene transcripts that are upregulated at 7 days post SRC as compared to baseline is shown in Table 5A. A list of gene transcripts that are downregulated at 7 days post SRC as compared to baseline is shown in Table 5B.

A list of gene transcripts that are differentially expressed at 6 hours post SRC and 7 days post SRC, as compared to baseline; and which are differentially expressed at 7 days post SRC as compared to 6 hours post SRC is shown in Table 6.

Differential Gene Expression Before and after SRC

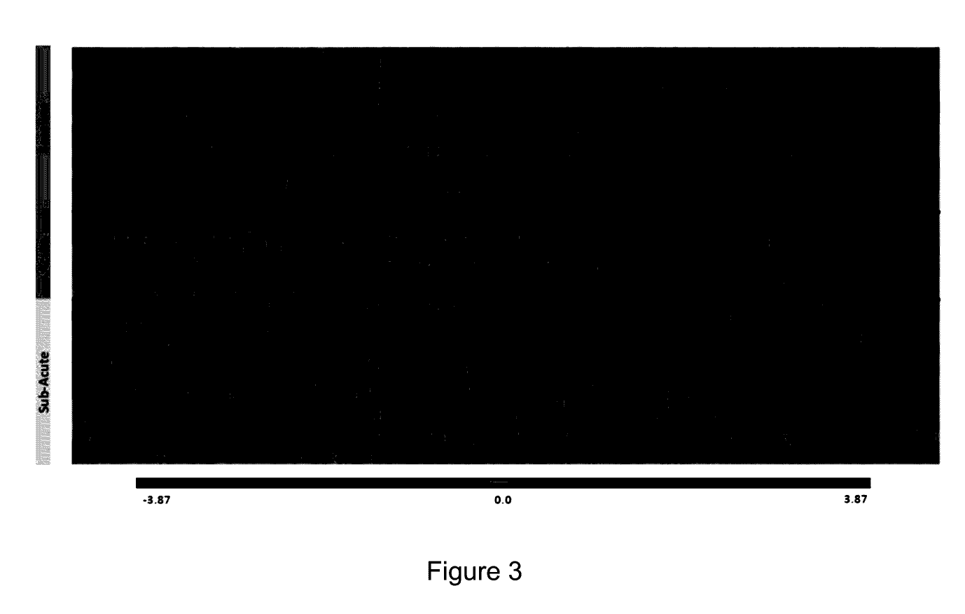

Among concussed athletes at baseline (i.e., before injury), the majority of differentially expressed gene transcripts displayed increased transcriptional activity (FIG. 3). However, acutely after SRC, of the 593 probesets that were differentially expressed, the majority (435, 73%) were down-regulated. This pattern persisted into the sub-acute time point, where 408 of the 479 (85%) differentially expressed gene transcripts were down-regulated. Three hundred and nine transcripts were differentially expressed at both the acute and sub-acute time points.

Canonical Pathways and Biologic Networks Associated with Changes in Gene Expression after SRC

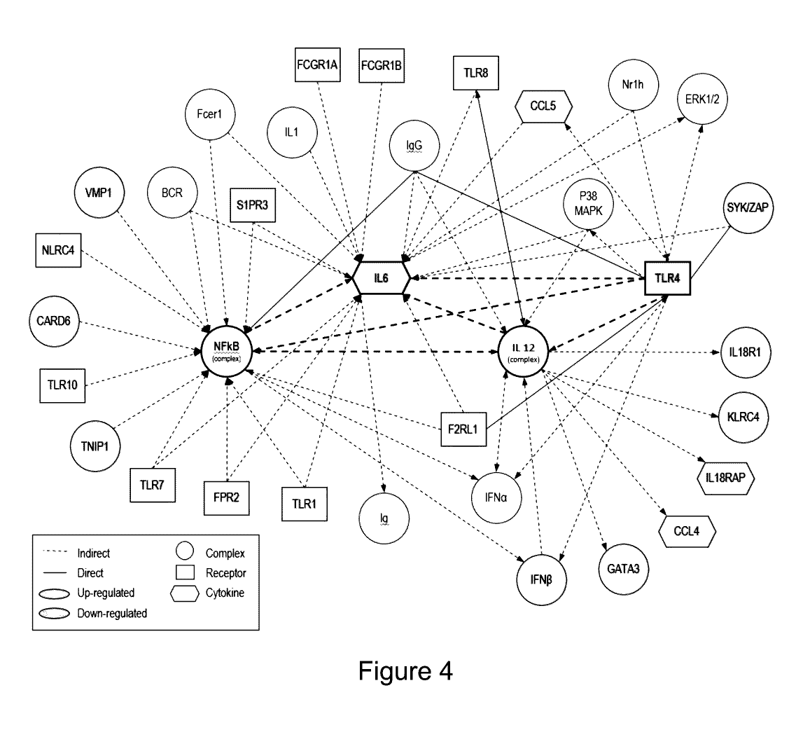

The top canonical pathways of genes that displayed significant changes from baseline to the acute post-SRC time point involved ‘Immune Cell Functioning’ and ‘Communication’ that included the NF-κB and natural killer signaling pathways. The top network of gene changes were related to ‘Inflammatory Response, Infectious Disease, and Renal/Urological Disease’ (FIG. 4). Genes identified as hubs in this network were interleukin 6 (IL-6, 19 connections), NF-κB (18 connections), interleukin 12 (IL-12, 13 connections), and toll-like receptor 4 (TRL4, 13 connections).

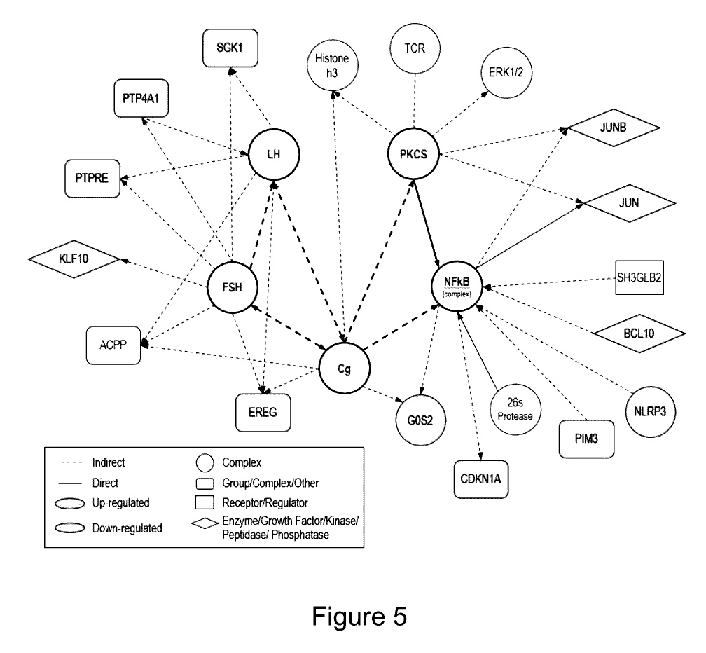

The top canonical pathways of genes that displayed significant changes from baseline to the sub-acute post-SRC time point involved ‘Glucocorticoid Receptor Signaling’, and the top network of gene changes were related to ‘Neurological Disease, Cell Death and Survival’ (FIG. 5). Genes identified as hubs in this network were NF-κB (11 connections), follicle-stimulating hormone (FSH, 8 connections), chorionic gonadotropin (Cg, 8 connections), luteinizing hormone (LH, 7 connections), and protein kinase catalytic subunit (PKCS, 7 connections).

Gene Expression Changes after TBI

The present study is believed to be the first study to describe temporal changes in networks of altered genes after SRC, and indeed, after human TBI of any severity. Others have reported longitudinal changes in gene expression following experimental TBI in rodents. In these studies, early changes were related to transcriptional regulation, inflammation and cell signaling; later changes were related to complement system major histocompatibility complex (MHC) class II pathway, and cell death/survival (Almeida-Suhett et al., 2014, J Neurotrauma, 31: 683-690; Redell et al., 2013, J Neurotrauma, 30: 752-764; Li et al., 2004, J Neurotrauma, 21: 1141-1153; Natale et al., 2003, J Neurotrauma, 20: 907-927).

Determining molecular causality and response to concussion is complex, but the present findings suggest a distinct shift in gene expression activity between the acute and the sub-acute post-concussion periods. Because all SRC athletes had recovered clinically back to baseline performance, these gene changes are interpreted to be adaptive. The cascade of molecular changes during the first 6 hours following SRC is dominated by inflammatory activity centered around IL-6, IL-12, toll-like receptors (TLR), and NF-κB. What these four hub genes have in common is their effect on regulating the innate immune response as well promoting the transition to an acquired, adaptive immune response.

Although the brain was once considered immunologically privileged, it is now known that it actively participates in inflammatory processes necessary for maintaining neural homeostasis. After experimental TBI, molecules such as heat shock proteins, high mobility group box-1 (HMGB-1), and hyaluronan released from damaged neurons have been shown to activate resident microglia via surface TLR (Park et al., 2008, Neuroscience Letters, 431: 123-128; Babcock et al., 2006, Journal of Neuroscience, 26: 12826-12837). TLRs are pattern recognition receptors that play an important role in the initiation of innate immunity (Lehnardt, 2010, Glia, 58: 253-263). Using a similar mRNA pathway analysis, TLR signaling was found to be an important transcriptional hub 3 hours after fluid percussion injury and 24 hours after controlled cortical impact in rats (Redell et al., 2013, J Neurotrauma, 30: 752-764).

NF-κB is a major transcription factor that regulates genes responsible for both the innate and adaptive immune response. In support of the present findings, two prior pathway analyses of mRNA expression in experimentally injured rodent brains also identified NF-κB signaling as an important transcriptional hub after TBI (Redell et al., 2013, J Neurotrauma, 30: 752-764; White et al., 2013, BMC Genomics, 14:282). NF-κB is known to be activated by stimulation of TLRs (Hayden et al., 2006, Oncogene, 25:6758-6780), which may explain why both were identified as key hubs after SRC. Moreover, downstream of NF-κB activation leads to expression of cytokines like IL-6 (Libermann and Baltimore, 1990, Molecular & Cellular Biology, 10: 2327-2334) and IL-12 (Murphy et al., 1995, Molecular & Cellular Biology, 15: 5258-5267). In the setting of more severe TBI, NF-κB expression has been found to be up-regulated in rodent models (Yang et al., 1995, Neuroscience Letters, 197: 101-104; Nonaka et al., 1999, J Neurotrauma, 16: 1023-1034; Sanz et al., 2002, Journal of Neuroscience Research, 67: 772-780) as well as in humans (Hang et al., 2006, Brain Research, 1109: 14-21). The precise role of NF-κB expression in modulating the innate and adaptive immune response after TBI has not been elucidated. However, genetically altered mice unable to up-regulate NF-κB have larger lesion volumes and blood-brain barrier breech after experimental TBI, suggesting that NF-κB activation may serve a neuroprotective function (Sullivan et al., 1999, Journal of Neuroscience, 19: 6248-6256) in this capacity.

IL-6 is an inflammatory cytokine that regulates the transition from innate to acquired immunity. The hallmark of this shift is a transition in the composition of inflammatory cells from neutrophils to mononuclear cells. IL-6 coordinates this transition by impacting cellular events that dampen innate immunity (e.g., suppressing chemokine release and promoting neutrophil apoptosis) while simultaneously promoting acquired immunity (e.g., promoting T-cell adhesion and blocking T-cell apoptosis) (Jones, 2005, Journal of Immunolgy, 175: 3463-3468). Several animal studies have demonstrated that TBI results in up-regulation of IL-6, and that a functioning IL-6 gene is necessary for recovery (Erta et al., 2012, International Journal of Biological Sciences, 8: 1254-1266). In fact, a functional polymorphism (−174C/G) in the promoter region of IL-6 was found to be associated with increased mortality after severe TBI in humans (Dalla Libera et al., 2011, Brain Injury, 25: 365-369). Using a mRNA pathway analysis, Redell et al. found the IL-6 signaling pathway to be an important hub within 3 hours of both controlled cortical impact and fluid percussion injury in mice (Redell et al., 2013, J Neurotrauma, 30: 752-764). As with animal studies, several human studies have shown that raised CSF levels of IL-6 correlate with improved post-TBI outcomes (Helmy et al., 2011, Journal of Cerebral Blood Flow & Metabolism, 31: 658-670).

Like IL-6, IL-12 is also a pro-inflammatory cytokine that participates in both innate (e.g., by inducing IFN-γ) and adaptive (e.g., by inducing a TH1 response in CD4+ cells) immunity (Trinchieri, 2003, Nature Reviews Immunology, 3: 133-146). Two human studies have reported elevated IL-12 in the CSF and interstitial fluid after severe TBI (Helmy et al., 2011, Journal of Cerebral Blood Flow & Metabolism, 31: 658-670; Stahel et al., 1998, Neuroscience Letters, 249: 123-126). Although its role in TBI is less clear, IL-12 may function to shift microglial activation from the pro-inflammatory M1 phenotype (where IL-12 expression is typically high) to the anti-inflammatory M2 phenotype (where expression is reduced). In support of this idea, naturally-occurring (Schwulst et al., 2013, The Journal of Trauma and Acute Care Surgery, 75: 780-788) and pharmacologically-induced (Gatson et al., 2012, The Journal of Trauma and Acute Care Surgery, 74: 470-474, discussion 474-475) reduction in IL-12 have been shown to be associated with reduced microglial activation after experimental TBI in mice.

Taken together, these findings suggest that in the acute phase, regulation of the innate immune response, and the transition to acquired immunity, is important for recovery from SRC. These results further suggest that 7 days post-injury, gene transcriptional activity shifts away from acute inflammation and toward the regulation of the hypothalamic-pituitary-adrenal (HPA) axis. Gene changes centering on glucocorticoid receptor signaling were observed, with NF-κB, FSH, LH, chorionic gonadotropin (Cg) and PKCS being the key transcriptional hubs. It is well known that more severe forms of TBI can disrupt the HPA axis, with reductions in growth hormone (GH) being the most commonly reported perturbation in humans (Schneider et al., 2011, J Neurotrauma, 28: 1693-1698). No prior reports have linked SRC to disturbances in the HPA axis, although retired boxers, kickboxers and professional football players have been shown to have various degrees of anterior pituitary dysfunction (Kelestimur et al., 2004, Journal of Endocrinological Investigation, 27: RC28-32; Tanriverdi et al., 2007, Clinical Endocrinology, 66: 360-366). Pre-clinical studies suggest that excessive glutamate receptor activation post-TBI impacts glucocorticoid mRNA transcription leading to its preferential down regulation, especially in the hippocampus (McCullers et al., 2002, Brain Research, 947: 41-49). Similar observations of the mesocorticolimbic system have been observed whereby excessive glucocorticoid receptor activation by cortisol increases the vulnerability of hippocampal neurons to damage from oxidative stress and excitiotoxicity (McCullers et al., 2001, J Neuroscience, 109: 219-230; McIntosh and Sapolsky, 1996, Neurotoxicity, 17: 873-882; Goodman et al., 1996, Journal of Neurochemistry, 66: 1836-1844). Thus, while not wishing to be bound by any particular throry, down-regulation of glucocorticoid receptor signaling may serve to protect these vulnerable neurons during the sub-acute period of recovery. In the process of protecting these neurons, however, this down-regulation may reduce the production of important hormones such as GH, FSH and LH, which can contribute to a variety of post-concussion symptoms. These gene activities are likely necessary to mitigate the excessive inflammation that develops during the acute period and present a shift in gene expression from neuronal proliferation to neuronal recovery, but may come at the expense of dysregulated hormonal control.

It was observed that the majority of gene transcripts were down regulated after SRC; 73% in the acute time period, and 85% at the sub-acute time period relative to baseline. This finding is in contradistinction to those in humans with severe TBI and many animal studies, where upregulation is more common. However, preclinical studies using rodent models of mTBI suggests that less severe brain injuries are associated with down as opposed to up regulation (Li et al., 2004, J Neurotrauma, 21: 1141-1153). Because all concussed athletes recovered clinically, suppression of inflammation and cell death cycles may be adaptive.

In summary, the present studies detected acute changes in peripheral gene expression following SRC, reflecting regulation of the innate immune response as well as the transition to an acquired, adaptive immune response. By 7 days post-injury, transcriptional activity is centered on the regulation of the HPA axis. These findings illustrate a time-dependent shift in gene expression post-injury that may provide insight into the pathophysiology of recovery from SRC.

TABLE 4A
Up-regulated differentially expressed transcripts: 6 hours post SRC (T2)
versus baseline (T1)

| | | Fold-Change |
|---|---|---|
| Gene Title | Gene Symbol | (T2 vs. T1) |
| ADAM metallopeptidase domain 9 | ADAM9 | 1.82176 |
| ADAM metallopeptidase with thrombospondin | ADAMTS5 | 1.83321 |
| type 1 motif, 5 | | |
| ADAM metallopeptidase with thrombospondin | ADAMTS5 | 2.18224 |
| type 1 motif, 5 | | |
| ADP-ribosylarginine hydrolase | ADPRH | 1.53673 |
| ankyrin repeat domain 57 | ANKRD57 | 1.56891 |
| adenomatosis polyposis coli down-regulated 1 | APCDD1 | 1.52647 |
| ATPase, H+ transporting, lysosomal 70 kDa, V1 | ATP6V1A | 1.77021 |
| subunit A | | |
| BTB and CNC homology 1, basic leucine zipper | BACH1 | 1.60519 |
| transcription factor 1 | | |
| bone marrow stromal cell antigen 1 | BST1 | 1.5328 |
| caspase recruitment domain family, member 6 | CARD6 | 1.53131 |
| chemokine (C-C motif) receptor 1 | CCR1 | 1.51571 |
| chemokine (C-C motif) receptor 1 | CCR1 | 1.60056 |
| chemokine (C-C motif) receptor-like 2 | CCRL2 | 1.51567 |
| CD93 molecule | CD93 | 1.50333 |
| CDC42 effector protein (Rho GTPase binding) 3 | CDC42EP3 | 1.50849 |
| CDC42 effector protein (Rho GTPase binding) 3 | CDC42EP3 | 1.53614 |
| CENPB DNA-binding domains containing 1 | CENPBD1 | 1.63975 |
| chloride intracellular channel 4 | CLIC4 | 1.56137 |
| chronic lymphocytic leukemia up-regulated 1 | CLLU1 | 1.65206 |
| carboxypeptidase D | CPD | 1.58561 |
| complement component (3b/4b) receptor 1 | CR1 | 1.52311 |
| (Knops blood group) | | |
| CTTNBP2 N-terminal like | CTTNBP2NL | 1.6428 |
| cytochrome P450, family 1, subfamily B, | CYP1B1 | 1.76538 |
| polypeptide 1 | | |
| cytochrome P450, family 1, subfamily B, | CYP1B1 | 1.83233 |
| polypeptide 1 | | |
| cytochrome P450, family 1, subfamily B, | CYP1B1 | 1.99311 |
| polypeptide 1 | | |
| dachshund homolog 1 (Drosophila) | DACH1 | 1.51638 |
| dual adaptor of phosphotyrosine and 3- | DAPP1 | 1.68966 |
| phosphoinositides | | |
| desmocollin 2 | DSC2 | 1.69681 |
| epithelial membrane protein 1 | EMP1 | 1.8738 |
| ectonucleoside triphosphate diphosphohydrolase 1 | ENTPD1 | 1.62899 |
| coagulation factor II (thrombin) receptor-like 1 | F2RL1 | 1.59557 |
| coagulation factor V (proaccelerin, labile factor) | F5 | 1.51541 |
| family with sequence similarity 114, member A1 | FAM114A1 | 1.63805 |
| family with sequence similarity 13, member A | FAM13A | 1.50801 |
| family with sequence similarity 198, member B | FAM198B | 2.22299 |
| fatty acyl CoA reductase 1 | FAR1 | 1.59784 |
| fatty acyl CoA reductase 2 | FAR2 | 1.68735 |
| fibrillin 2 | FBN2 | 1.721 |
| F-box protein 30 | FBXO30 | 1.57898 |
| Fc fragment of IgG, high affinity Ia, receptor | FCGR1A; FCGR1C | 1.76282 |
| (CD64); Fc fragment of IgG, high affi | | |
| Fc fragment of IgG, high affinity Ib, receptor | FCGR1B | 1.72442 |
| (CD64) | | |
| FK506 binding protein 15, 133 kDa | FKBP15 | 1.50442 |
| folate receptor 3 (gamma) | FOLR3 | 2.05781 |
| FOS-like antigen 2 | FOSL2 | 1.57091 |
| formyl peptide receptor 2 | FPR2 | 1.77041 |
| formyl peptide receptor 2 | FPR2 | 1.85476 |
| frequently rearranged in advanced T-cell | FRAT2 | 1.72608 |
| lymphomas 2 | | |
| far upstream element (FUSE) binding protein 1 | FUBP1 | 1.55437 |
| growth arrest-specific 7 | GAS7 | 1.52312 |
| G patch domain containing 2 | GPATCH2 | 1.61607 |
| G patch domain containing 2 | GPATCH2 | 1.70199 |
| G protein-coupled receptor 27 | GPR27 | 1.53487 |
| hematopoietically expressed homeobox | HHEX | 1.51675 |
| hematopoietically expressed homeobox | HHEX | 1.7329 |
| haptoglobin | HP | 1.72582 |
| interferon gamma receptor 1 | IFNGR1 | 1.51823 |
| potassium voltage-gated channel, Isk-related | KCNE3 | 1.62682 |
| family, member 3 | | |
| microfibrillar-associated protein 3A117:D117 | MFAP3 | 1.58049 |
| microsomal glutathione S-transferase 1 | MGST1 | 1.63932 |
| microsomal glutathione S-transferase 1 | MGST1 | 1.69246 |
| Microsomal glutathione S-transferase 1 | MGST1 | 1.69458 |
| microsomal glutathione S-transferase 1 | MGST1 | 1.74784 |
| microRNA 21 | MIR21 | 2.68409 |
| molybdenum cofactor synthesis 3 | MOCS3 | 1.5075 |
| NLR family, CARD domain containing 4 | NLRC4 | 1.5579 |
| NLR family, pyrin domain containing 12 | NLRP12 | 1.50986 |
| oligonucleotide/oligosaccharide-binding fold | OBFC2A | 1.52002 |
| containing 2A | | |
| oligonucleotide/oligosaccharide-binding fold | OBFC2A | 1.55822 |
| containing 2A | | |
| purinergic receptor P2Y, G-protein coupled, 13 | P2RY13 | 1.73405 |
| phosphodiesterase 7B | PDE7B | 1.51869 |
| pyruvate dehydrogenase kinase, isozyme 4 | PDK4 | 2.99062 |
| pyruvate dehydrogenase kinase, isozyme 4 | PDK4 | 3.1238 |
| PHD finger protein 23 | PHF23 | 1.53301 |
| phosphatidylinositol glycan anchor biosynthesis, | PIGM | 1.58922 |
| class M | | |
| phospholipase A2, group IVA (cytosolic, | PLA2G4A | 1.87895 |
| calcium-dependent) | | |
| phospholipase D1, phosphatidylcholine-specific | PLD1 | 1.69193 |
| protein tyrosine phosphatase, receptor type, O | PTPRO | 2.21369 |
| Ras and Rab interactor 2 | RIN2 | 1.62017 |
| ring finger protein 24 | RNF24 | 1.5887 |
| sphingosine-1-phosphate receptor 3 | S1PR3 | 1.57953 |
| SAP30-like | SAP30L | 1.599 |
| serpin peptidase inhibitor, clade I (pancpin), | SERPINI2 | 1.60236 |
| member 2 | | |
| sphingomyelin synthase 2 | SGMS2 | 1.76728 |
| Signal-induced proliferation-associated 1 like 1 | SIPA1L1 | 1.63837 |
| signal-regulatory protein beta 2 | SIRPB2 | 1.55351 |
| SLIT and NTRK-like family, member 4 | SLITRK4 | 2.16832 |
| spermatogenesis associated 5-like 1 | SPATA5L1 | 1.87522 |
| ST6 (alpha-N-acetyl-neuraminyl-2,3-beta- | ST6GALNAC3 | 1.70328 |
| galactosyl-1,3)-N-acetylgalactosaminide alpha-2 | | |
| ST8 alpha-N-acetyl-neuraminide alpha-2,8- | ST8SIA4 | 1.52364 |
| sialyltransferase 4 | | |
| ST8 alpha-N-acetyl-neuraminide alpha-2,8- | ST8SIA4 | 1.66224 |
| sialyltransferase 4 | | |
| STEAP family member 4 | STEAP4 | 2.01298 |
| transcription factor EC | TFEC | 1.75494 |
| toll-like receptor 1 | TLR1 | 1.53858 |
| toll-like receptor 10 | TLR10 | 1.54391 |
| toll-like receptor 4 | TLR4 | 1.54384 |
| toll-like receptor 4 | TLR4 | 1.66149 |
| toll-like receptor 7 | TLR7 | 1.59205 |
| toll-like receptor 8 | TLR8 | 1.62471 |
| transmembrane protein 49 | TMEM49 | 1.8221 |
| trichorhinophalangeal syndrome I | TRPS1 | 1.56073 |
| versican | VCAN | 1.70542 |
| von Willebrand factor A domain containing 5A | VWA5A | 1.59852 |
| wntless homolog (Drosophila) | WLS | 1.59272 |
| wntless homolog (Drosophila) | WLS | 1.76165 |
| WD repeat and SOCS box-containing 1 | WSB1 | 1.82444 |
| zinc finger E-box binding homeobox 2 | ZEB2 | 1.61326 |
| zinc finger protein 322B | ZNF322B | 1.60589 |
| zinc finger protein 697 | ZNF697 | 1.63815 |
| zinc finger protein 780A | ZNF780A | 1.53325 |

TABLE 4B
Down-regulated differentially expressed transcripts: 6 hours post SRC (T2) vs.
Baseline (T1)

| | | Fold-Change |
|---|---|---|
| Gene Title | Gene Symbol | (T2 vs. T1) |
| adenosine deaminase | ADA | −1.72356 |
| ArfGAP with GTPase domain, ankyrin repeat | AGAP1 | −1.80837 |
| and PH domain 1 | | |
| ankyrin repeat domain 36 | ANKRD36 | −1.63932 |
| ADP-ribosylation factor-like 4C | ARL4C | −1.8832 |
| ADP-ribosylation factor-like 4C | ARL4C | −1.87226 |
| ADP-ribosylation factor-like 4C | ARL4C | −1.85424 |
| ATPase, class VI, type 11B | ATP11B | −1.50127 |
| autism susceptibility candidate 2 | AUTS2 | −1.7547 |
| benzodiazapine receptor (peripheral) associated | BZRAP1 | −1.84583 |
| protein 1 | | |
| Calumenin | CALU | −1.50768 |
| Cas-Br-M (murine) ecotropic retroviral | CBLB | −1.5979 |
| transforming sequence b | | |
| chemokine (C-C motif) ligand 4 | CCL4 | −3.38991 |
| chemokine (C-C motif) ligand 5 | CCL5 | −1.56621 |
| chemokine (C-C motif) ligand 5 | CCL5 | −1.56409 |
| CD96 molecule | CD96 | −2.32104 |
| CDC14 cell division cycle 14 homolog A (S. cerevisiae) | CDC14A | −2.22332 |
| centrosomal protein 78 kDa | CEP78 | −1.88364 |
| Chromodomain helicase DNA binding protein 2 | CHD2 | −1.70091 |
| catenin (cadherin-associated protein), beta 1, | CTNNB1 | −1.6076 |
| 88 kDa | | |
| cathepsin W | CTSW | −1.88397 |
| deltex homolog 3 (Drosophila) | DTX3 | −1.70068 |
| EH-domain containing 4 | EHD4 | −1.50216 |
| eukaryotic translation initiation factor 1 | EIF1 | −1.52229 |
| EP400 N-terminal like | EP400NL | −1.6405 |
| family with sequence similarity 100, member B | FAM100B | −1.53662 |
| family with sequence similarity 179, member A | FAM179A | −1.68265 |
| fibroblast growth factor binding protein 2 | FGFBP2 | −1.95856 |
| fused in sarcoma | FUS | −1.52544 |
| GATA binding protein 3 | GATA3 | −2.29336 |
| guanylate binding protein 5 | GBP5 | −1.67871 |
| growth factor independent 1 transcription | GFI1 | −1.73194 |
| repressor | | |
| glucocorticoid induced transcript 1 | GLCCI1 | −1.57928 |
| GrpE-like 1, mitochondrial (E. coli) | GRPEL1 | −1.53133 |
| granzyme B (granzyme 2, cytotoxic T- | GZMB | −2.07291 |
| lymphocyte-associated serine esterase 1) | | |
| granzyme H (cathepsin G-like 2, protein h- | GZMH | −2.10351 |
| CCPX) | | |
| granzyme M (lymphocyte met-ase 1) | GZMM | −1.93125 |
| heterogeneous nuclear ribonucleoprotein L | HNRNPL | −1.5871 |
| homeobox B3 | HOXB3 | −1.63953 |
| inhibitor of DNA binding 2, dominant negative | ID2 | −1.63656 |
| helix-loop-helix protein | | |
| inhibitor of DNA binding 2, dominant negative | ID2 | −1.60803 |
| helix-loop-helix protein | | |
| interferon induced transmembrane protein 1 (9- | IFITM1 | −1.67642 |
| 27) | | |
| interferon induced transmembrane protein 1 (9- | IFITM1 | −1.59115 |
| 27) | | |
| interleukin 18 receptor 1 | IL18R1 | −1.79239 |
| interleukin 18 receptor accessory protein | IL18RAP | −1.90962 |
| interleukin 6 (interferon, beta 2) | IL6 | −1.66587 |
| KIAA1671 | KIAA1671 | −1.6961 |
| kinesin family member 21A | KIF21A | −1.60799 |
| killer cell immunoglobulin-like receptor, three | KIR3DL1; KIR3DL2 | −1.79571 |
| domains, long cytoplasmic tail, 1; k | | |
| killer cell immunoglobulin-like receptor, three | KIR3DL3 | −1.70915 |
| domains, long cytoplasmic tail, 3 | | |
| Kruppel-like factor 12 | KLF12 | −2.06742 |
| killer cell lectin-like receptor subfamily B, | KLRB1 | −1.90964 |
| member 1 | | |
| killer cell lectin-like receptor subfamily D, | KLRD1 | −1.86593 |
| member 1 | | |
| killer cell lectin-like receptor subfamily K, | KLRK1 | −1.78448 |
| member 1 | | |
| Muscleblind-like 2 (Drosophila) | MBNL2 | −2.06215 |
| Mannosyl (alpha-1,3-)-glycoprotein beta-1,4-N- | MGAT4A | −2.54034 |
| acetylglucosaminyltransferase, isozyme A | | |
| mannosyl (alpha-1,3-)-glycoprotein beta-1,4-N- | MGAT4A | −1.67906 |
| acetylglucosaminyltransferase, isozyme A | | |
| megalencephalic leukoencephalopathy with | MLC1 | −1.66993 |
| subcortical cysts 1 | | |
| myeloid/lymphoid or mixed-lineage leukemia | MLLT11 | −1.59317 |
| (trithorax homolog, Drosophila); translocate | | |
| v-myb myeloblastosis viral oncogene homolog | MYBL1 | −2.14559 |
| (avian)-like 1 | | |
| myosin regulatory light chain interacting protein | MYLIP | −1.89332 |
| myosin regulatory light chain interacting protein | MYLIP | −1.50894 |
| nucleosome assembly protein 1-like 5 | NAP1L5 | −1.9825 |
| neural cell adhesion molecule 1 | NCAM1 | −1.68845 |
| nuclear factor of activated T-cells, cytoplasmic, | NFATC2 | −1.77617 |
| calcineurin-dependent 2 | | |
| nuclear factor of activated T-cells, cytoplasmic, | NFATC2 | −1.63898 |
| calcineurin-dependent 2 | | |
| nucleoporin 153 kDa | NUP153 | −1.52758 |
| Poly(rC) binding protein 2 | PCBP2 | −1.64274 |
| phosphodiesterase 4B, cAMP-specific | PDE4B | −1.55035 |
| phosphodiesterase 4D, cAMP-specific | PDE4D | −1.53463 |
| period homolog 1 (Drosophila) | PER1 | −2.34547 |
| period homolog 1 (Drosophila) | PER1 | −1.70146 |
| phosphoinositide-3-kinase, regulatory subunit 1 | PIK3R1 | −1.56311 |
| (alpha) | | |
| protein kinase C, eta | PRKCH | −1.68436 |
| protein kinase C, theta | PRKCQ | −1.86552 |
| proline rich 5 (renal) | PRR5 | −1.81251 |
| pituitary tumor-transforming 1 | PTTG1 | −1.52511 |
| pyrin and HIN domain family, member 1 | PYHIN1 | −1.93462 |
| RasGEF domain family, member 1A | RASGEF1A | −1.60149 |
| RNA binding motif protein 14 | RBM14 | −1.54638 |
| RAR-related orphan receptor A | RORA | −2.68628 |
| RAR-related orphan receptor A | RORA | −2.63409 |
| runt-related transcription factor 3 | RUNX3 | −1.77892 |
| runt-related transcription factor 3 | RUNX3 | −1.69184 |
| sphingosine-1-phosphate receptor 5 | S1PR5 | −1.99987 |
| sterile alpha motif domain containing 3 | SAMD3 | −1.81295 |
| sestrin 2 | SESN2 | −1.57849 |
| sestrin 2 | SESN2 | −1.56417 |
| SH2 domain containing 2A | SH2D2A | −1.80828 |
| spondin 2, extracellular matrix protein | SPON2 | −2.06256 |
| spectrin, beta, non-erythrocytic 1 | SPTBN1 | −1.63193 |
| spectrin repeat containing, nuclear envelope 2 | SYNE2 | −2.07536 |
| spectrin repeat containing, nuclear envelope 2 | SYNE2 | −1.63663 |
| synaptotagmin-like 2 | SYTL2 | −1.85889 |
| TAF9B RNA polymerase II, TATA box binding | TAF9B | −1.5885 |
| protein (TBP)-associated factor, 31 kDa | | |
| TCR gamma alternate reading frame protein | TARP | −2.2425 |
| TCR gamma alternate reading frame protein; T | TARP; TRGC2 | −2.31319 |
| cell receptor gamma constant 2 | | |
| TCR gamma alternate reading frame protein; T | TARP; TRGC2 | −2.18635 |
| cell receptor gamma constant 2 | | |
| TCR gamma alternate reading frame protein; T | TARP; TRGC2 | −2.17218 |
| cell receptor gamma constant 2 | | |
| transcription factor Dp-2 (E2F dimerization | TFDP2 | −1.55293 |
| partner 2) | | |
| transforming growth factor, beta receptor III | TGFBR3 | −1.8931 |
| T cell immunoreceptor with Ig and ITIM | TIGIT | −1.79421 |
| domains | | |
| TRAF2 and NCK interacting kinase | TNIK | −1.59894 |
| T cell receptor delta locus | TRD | −1.95283 |
| T cell receptor delta locus | TRD | −1.9207 |
| TSC22 domain family, member 3 | TSC22D3 | −1.91096 |
| TSC22 domain family, member 3 | TSC22D3 | −1.81404 |
| tRNA splicing endonuclease 54 homolog (S. cerevisiae) | TSEN54 | −1.60547 |
| tRNA splicing endonuclease 54 homolog (S. cerevisiae) | TSEN54 | −1.50904 |
| tetraspanin 5 | TSPAN5 | −1.60208 |
| ubiquitin-conjugating enzyme E2B (RAD6 | UBE2B | −1.81879 |
| homolog) | | |
| ubiquitin-conjugating enzyme E2I (UBC9 | UBE2I | −1.66093 |
| homolog, yeast) | | |
| WW domain containing adaptor with coiled-coil | WAC | −1.52328 |
| zeta-chain (TCR) associated protein kinase | ZAP70 | −1.89061 |
| 70 kDa | | |
| zinc finger protein 451 | ZNF451 | −1.60463 |
| zinc finger protein 507 | ZNF507 | −1.52977 |
| zinc finger protein 831 | ZNF831 | −2.21191 |
| zinc finger protein 831 | ZNF831 | −2.07393 |

TABLE 5A
Up-regulated differentially expressed transcripts:
7 day post SRC (T5) versus baseline (T1)

| | Gene | Fold-Change |
|---|---|---|
| Gene Title | Symbol | (T5 vs. T1) |
| ATP synthase, H+ transporting, mitochondrial | ATP5C1 | 1.90782 |
| F1 complex, gamma polypeptide 1 | | |
| bromodomain and WD repeat domain | BRWD1 | 1.50976 |
| containing 1 | | |
| cell division cycle 37 homolog | CDC37L1 | 1.57447 |
| (S. cerevisiae)-like 1 | | |
| cold inducible RNA binding protein | CIRBP | 1.72452 |
| chemokine (C-X3-C motif) receptor 1 | CX3CR1 | 1.906 |
| DTW domain containing 1 | DTWD1 | 1.58646 |
| EPM2A (laforin) interacting protein 1 | EPM2AIP1 | 2.06515 |
| lysosomal trafficking regulator | LYST | 1.6137 |
| N-acyl phosphatidylethanolamine | NAPEPLD | 1.58975 |
| phospholipase D | | |
| N-acetylneuraminate pyruvate lyase | NPL | 1.70268 |
| (dihydrodipicolinate synthase) | | |
| ras homolog gene family, member U | RHOU | 1.50428 |
| Ribosomal protein S24 | RPS24 | 1.92439 |
| remodeling and spacing factor 1 | RSF1 | 1.52332 |
| SMAD family member 4 | SMAD4 | 1.67394 |
| TATA box binding protein (TBP)-associated | TAF1D | 1.61806 |
| factor, RNA polymerase I, D, 41 kDa | | |
| tripartite motif-containing 13 | TRIM13 | 1.66186 |
| tetratricopeptide repeat domain 9C | TTC9C | 1.52731 |
| zinc finger protein 253 | ZNF253 | 1.51259 |
| Zinc finger protein 302 | ZNF302 | 1.51828 |
| zinc finger protein 451 | ZNF451 | 1.68414 |
| zinc finger protein 557 | ZNF557 | 1.53981 |
| zinc finger protein 594 | ZNF594 | 1.51292 |
| zinc finger protein 606 | ZNF606 | 1.67745 |
| Zinc finger protein 641 | ZNF641 | 1.52191 |

TABLE 5B
Down-regulated differentially expressed transcripts:
7 day post SRC (T5) versus baseline (T1)

| | Gene | Fold-Change |
|---|---|---|
| Gene Title | Symbol | (T5 vs. T1) |
| ankyrin repeat domain 13C | ANKRD13C | −1.58842 |
| amyloid beta (A4) precursor protein-binding, | APBB1IP | −1.66075 |
| family B, member 1 interacting protein | | |
| additional sex combs like 1 (Drosophila) | ASXL1 | −1.52125 |
| B-cell CLL/lymphoma 10 | BCL10 | −2.19636 |
| chromosome 15 open reading frame 39 | C15orf39 | −1.6475 |
| chromosome 15 open reading frame 48 | C15orf48 | −1.76357 |
| chromosome 7 open reading frame 40 | C7orf40 | −1.56944 |
| chromosome 9 open reading frame 72 | C9orf72 | −1.99432 |
| chromosome 9 open reading frame 72 | C9orf72 | −1.66786 |
| chemokine (C-C motif) ligand 3; chemokine | CCL3; | −5.99786 |
| (C-C motif) ligand 3-like 1; chemokine | CCL3L1 | |
| cyclin L1 | CCNL1 | −2.07154 |
| cytidine and dCMP deaminase domain | CDADC1 | −1.5394 |
| containing 1 | | |
| cell division cycle 42 (GTP binding protein, | CDC42 | −1.76237 |
| 25 kDa) | | |
| cyclin-dependent kinase inhibitor 1A | CDKN1A | −1.93681 |
| (p21, Cip1) | | |
| Cullin 1 | CUL1 | −1.66985 |
| DNA-damage-inducible transcript 3 | DDIT3 | −1.77049 |
| dicer 1, ribonuclease type III | DICER1 | −1.96647 |
| dual specificity phosphatase 1 | DUSP1 | −2.89657 |
| dual specificity phosphatase 1 | DUSP1 | −1.69933 |
| early growth response 2 | EGR2 | −2.74073 |
| early growth response 3 | EGR3 | −3.33259 |
| eukaryotic translation initiation factor 1 | EIF1 | −2.12949 |
| Eukaryotic translation initiation factor 4A1 | EIF4A1 | −1.54614 |
| epiregulin | EREG | −1.95429 |
| ets variant 3 | ETV3 | −1.85529 |
| Fc fragment of IgA, receptor for | FCAR | −2.03608 |
| fem-1 homolog b (C. elegans) | FEM1B | −1.73778 |
| free fatty acid receptor 2 | FFAR2 | −2.1404 |
| G0/G1 switch 2 | G0S2 | −7.72302 |
| growth arrest and DNA-damage-inducible, beta | GADD45B | −1.61155 |
| growth arrest and DNA-damage-inducible, beta | GADD45B | −1.57904 |
| growth arrest and DNA-damage-inducible, beta | GADD45B | −1.55575 |
| guanine nucleotide binding protein (G protein), | GNA13 | −1.5755 |
| alpha 13 | | |
| heterogeneous nuclear ribonucleoprotein M | HNRNPM | −1.55106 |
| immediate early response 2 | IER2 | −1.73493 |
| immediate early response 3 | IER3 | −1.96872 |
| interferon-related developmental regulator 1 | IFRD1 | −2.02534 |
| interleukin 1, beta | IL1B | −3.19348 |
| interleukin 1, beta | IL1B | −2.7545 |
| iron-responsive element binding protein 2 | IREB2 | −1.51584 |
| jun proto-oncogene | JUN | −4.72979 |
| jun proto-oncogene | JUN | −3.89976 |
| jun proto-oncogene | JUN | −3.50822 |
| Jun oncogene | JUN | −2.95401 |
| jun B proto-oncogene | JUNB | −1.74004 |
| kelch repeat and BTB (POZ) domain | KBTBD2 | −1.69939 |
| containing 2 | | |
| kinesin family member 13A | KIF13A | −1.58988 |
| Kruppel-like factor 10 | KLF10 | −2.05281 |
| Kruppel-like factor 11 | KLF11 | −1.51716 |
| Kruppel-like factor 4 (gut) | KLF4 | −1.57311 |
| v-Ki-ras2 Kirsten rat sarcoma viral oncogene | KRAS | −2.04976 |
| homolog | | |
| LIM and senescent cell antigen-like domains 3 | LIMS3 | −1.51259 |
| M-phase phosphoprotein 8 | MPHOSPH8 | −1.53486 |
| myotubularin related protein 12 | MTMR12 | −1.56425 |
| MAX dimerization protein 1 | MXD1 | −2.33384 |
| Nicotinamide phosphoribosyltransferase | NAMPT | −4.0493 |
| nicotinamide phosphoribosyltransferase | NAMPT | −2.06156 |
| nuclear factor of kappa light polypeptide gene | NFKBID | −1.72533 |
| enhancer in B-cells inhibitor, delta | | |
| NLR family, pyrin domain containing 3 | NLRP3 | −1.77103 |
| nuclear receptor subfamily 4, group A, | NR4A1 | −1.86138 |
| member 1 | | |
| phosphodiesterase 4D interacting protein | PDE4DIP | −1.50867 |
| Pellino homolog 1 (Drosophila) | PELI1 | −1.54829 |
| phosphatase and actin regulator 1 | PHACTR1 | −2.52952 |
| pleckstrin homology-like domain, family A, | PHLDA2 | −2.73675 |
| member 2 | | |
| pim-3 oncogene | PIM3 | −1.59295 |
| polo-like kinase 2 | PLK2 | −2.01644 |
| phorbol-12 -myristate-13-acetate-induced | PMAIP1 | −1.79113 |
| protein 1 | | |
| prostaglandin-endoperoxide synthase 2 | PTGS2 | −3.16529 |
| (prostaglandin G/H synthase and | | |
| cyclooxygenase) | | |
| prostaglandin-endoperoxide synthase 2 | PTGS2 | −2.86159 |
| (prostaglandin G/H synthase and | | |
| cyclooxygenase) | | |
| protein tyrosine phosphatase type IVA, | PTP4A1 | −1.73549 |
| member 1 | | |
| protein tyrosine phosphatase, receptor type, E | PTPRE | −1.6503 |
| 6-pyruvoyltetrahydropterin synthase | PTS | −1.53417 |
| purine-rich element binding protein B | PURB | −1.60848 |
| purine-rich element binding protein B | PURB | −1.56925 |
| ras homolog gene family, member Q | RHOQ | −1.6459 |
| ribosomal protein S16 | RPS16 | −1.50734 |
| Splicing factor, arginine/serine-rich 15 | SFRS15 | −1.72389 |
| Splicing factor, arginine/serine-rich 3 | SFRS3 | −1.55567 |
| serum/glucocorticoid regulated kinase 1 | SGK1 | −1.66063 |
| SIVA1, apoptosis-inducing factor | SIVA1 | −1.60965 |
| SMEK homolog 2, suppressor of mek1 | SMEK2 | −1.61728 |
| (Dictyostelium) | | |
| small nucleolar RNA, H/ACA box 68 | SNORA68 | −1.5639 |
| Small nuclear ribonucleoprotein polypeptide A′ | SNRPA1 | −1.65857 |
| sorting nexin 5 | SNX5 | −1.50432 |
| suppressor of cytokine signaling 3 | SOCS3 | −2.11105 |
| segue sto some 1 | SQSTM1 | −1.7724 |
| serine/arginine-rich splicing factor 5 | SRSF5 | −1.79767 |
| signal transducer and activator of transcription 3 | STAT3 | −2.17143 |
| (acute-phase response factor) | | |
| TAF5-like RNA polymerase II, p300/CBP- | TAF5L | −1.60064 |
| associated factor (PCAF)-associated factor, | | |
| 65 kDa | | |
| t-complex 11 (mouse)-like 2 | TCP11L2 | −1.58048 |
| transmembrane protein 107 | TMEM107 | −2.26695 |
| tumor necrosis factor | TNF | −2.90578 |
| tumor protein p53 inducible nuclear protein 2 | TP53INP2 | −2.23152 |
| tripeptidyl peptidase II | TPP2 | −2.09592 |
| trichorhinophalangeal syndrome I | TRPS1 | −1.50273 |
| tubulin, beta 2A | TUBB2A | −2.46102 |
| ubiquitin-conjugating enzyme E2, J1 (UBC6 | UBE2J1 | −1.54134 |
| homolog, yeast) | | |
| Wilms tumor 1 associated protein | WTAP | −1.85625 |
| yrdC domain containing (E. coli) | YRDC | −1.50924 |
| zinc finger and BTB domain containing 24 | ZBTB24 | −2.51896 |
| zinc finger, AN1-type domain 5 | ZFAND5 | −1.55571 |

TABLE 6
Differentially expressed transcripts at both 6 hours post SRC (T2) and 7 day post SRC (T5) versus
baseline (T1) and differentially expressed at 7 days post SRC (T5) versus 6 hours post SRC (T2).

| | | Fold-Change | Fold-Change |
|---|---|---|---|
| Gene Title | Gene Symbol | (T5 vs. T2) | Description |
| ankyrin repeat domain 50 | ANKRD50 | −1.18931 | 5 down vs 2 |
| ankyrin repeat domain 50 | ANKRD50 | −1.08009 | 5 down vs 2 |
| amphiregulin | AREG | 1.32082 | 5 up vs 2 |
| Amphiregulin B | AREGB | 1.06473 | 5 up vs 2 |
| ADP-ribosylation factor 1 | ARF1 | −1.05144 | 5 down vs 2 |
| ADP-ribosylation factor-like 4A | ARL4A | −1.16869 | 5 down vs 2 |
| armadillo repeat containing 8 | ARMC8 | −1.16138 | 5 down vs 2 |
| additional sex combs like 1 (Drosophila) | ASXL1 | −1.01199 | 5 down vs 2 |
| additional sex combs like 1 (Drosophila) | ASXL1 | 1.04754 | 5 up vs 2 |
| activating transcription factor 3 | ATF3 | −1.03872 | 5 down vs 2 |
| Beta-2-microglobulin | B2M | 1.00114 | 5 up vs 2 |
| basic helix-loop-helix family, member e40 | BHLHE40 | 1.05656 | 5 up vs 2 |
| B-cell translocation gene 1, anti-proliferative | BTG1 | −1.17242 | 5 down vs 2 |
| BTG family, member 3 | BTG3 | 1.15672 | 5 up vs 2 |
| BTG family, member 3 | BTG3 | 1.19515 | 5 up vs 2 |
| Chromosome 13 open reading frame 15 | C13orf15 | 1.12755 | 5 up vs 2 |
| chromosome 14 open reading frame 181 | C14orf181 | 1.01134 | 5 up vs 2 |
| chromosome 17 open reading frame 91 | C17orf91 | −1.219 | 5 down vs 2 |
| chromosome 1 open reading frame 55 | C1orf55 | 1.01424 | 5 up vs 2 |
| chromosome 5 open reading frame 41 | C5orf41 | −1.18098 | 5 down vs 2 |
| cyclin L1 | CCNL1 | −1.01401 | 5 down vs 2 |
| cyclin L1 | CCNL1 | 1.03067 | 5 up vs 2 |
| chemokine (C-C motif) receptor 2 | CCR2 | −1.51354 | 5 down vs 2 |
| chemokine (C-C motif) receptor 2 | CCR2 | −1.10007 | 5 down vs 2 |
| CD36 molecule (thrombospondin | CD36 | −1.40935 | 5 down vs 2 |
| receptor) | | | |
| CD44 molecule (Indian blood group) | CD44 | −1.24129 | 5 down vs 2 |
| CD69 molecule | CD69 | 1.58057 | 5 up vs 2 |
| CD83 molecule | CD83 | −1.22535 | 5 down vs 2 |
| cell division cycle 42 (GTP binding | CDC42 | −1.03355 | 5 down vs 2 |
| protein, 25 kDa) | | | |
| cytoskeleton associated protein 2 | CKAP2 | −1.04875 | 5 down vs 2 |
| cyclin M2 | CNNM2 | 1.00987 | 5 up vs 2 |
| consortin, connexin sorting protein | CNST | 1.0164 | 5 up vs 2 |
| casein kinase 1, epsilon | CSNK1E | 1.1568 | 5 up vs 2 |
| cysteine-serine-rich nuclear protein 1 | CSRNP1 | −1.09933 | 5 down vs 2 |
| chemokine (C-X-C motif) ligand 2 | CXCL2 | −1.59062 | 5 down vs 2 |
| chemokine (C-X-C motif) receptor 4 | CXCR4 | 1.18355 | 5 up vs 2 |
| chemokine (C-X-C motif) receptor 4 | CXCR4 | 1.21425 | 5 up vs 2 |
| chemokine (C-X-C motif) receptor 4 | CXCR4 | 1.2396 | 5 up vs 2 |
| cytochrome c, somatic | CYCS | 1.05508 | 5 up vs 2 |
| cylindromatosis (turban tumor syndrome) | CYLD | 1.04052 | 5 up vs 2 |
| cylindromatosis (turban tumor syndrome) | CYLD | 1.08272 | 5 up vs 2 |
| DNA-damage-inducible transcript 4 | DDIT4 | 1.81582 | 5 up vs 2 |
| discs, large homolog 1 (Drosophila) | DLG1 | −1.09532 | 5 down vs 2 |
| DnaJ (Hsp40) homolog, subfamily B, | DNAJB1 | 1.18496 | 5 up vs 2 |
| member 1 | | | |
| dual specificity phosphatase 2 | DUSP2 | 1.24926 | 5 up vs 2 |
| dual specificity phosphatase 5 | DUSP5 | 1.23018 | 5 up vs 2 |
| dynein, light chain, LC8-type 2 | DYNLL2 | 1.00641 | 5 up vs 2 |
| EH-domain containing 4 | EHD4 | 1.0002 | 5 up vs 2 |
| eukaryotic translation initiation factor 1 | EIF1 | −1.00982 | 5 down vs 2 |
| eukaryotic translation initiation factor 1 | EIF1 | −1.0086 | 5 down vs 2 |
| eukaryotic translation initiation factor 1 | EIF1 | −1.00719 | 5 down vs 2 |
| eukaryotic translation initiation factor 4 | EIF4G3 | −1.07925 | 5 down vs 2 |
| gamma, 3 | | | |
| eukaryotic translation initiation factor 5 | EIF5 | 1.01045 | 5 up vs 2 |
| epiregulin | EREG | −1.08747 | 5 down vs 2 |
| endoplasmic reticulum to nucleus | ERN1 | 1.10734 | 5 up vs 2 |
| signaling 1 | | | |
| ethanolamine kinase 1 | ETNK1 | −1.08691 | 5 down vs 2 |
| eyes absent homolog 3 (Drosophila) | EYA3 | −1.04762 | 5 down vs 2 |
| family with sequence similarity 46, | FAM46C | 1.05078 | 5 up vs 2 |
| member C | | | |
| hypothetical protein FLJ10038 | FLJ10038 | 1.09113 | 5 up vs 2 |
| FBJ murine osteosarcoma viral oncogene | FOSB | 1.21621 | 5 up vs 2 |
| homolog B | | | |
| fucose-1-phosphate guanylyltransferase | FPGT | −1.14595 | 5 down vs 2 |
| ferritin, heavy polypeptide 1 | FTH1 | −1.13999 | 5 down vs 2 |
| GABA(A) receptor-associated protein | GABARAPL1 | −1.03835 | 5 down vs 2 |
| like 1 | | | |
| GABA(A) receptor-associated protein | GABARAPL1/// | 1.07409 | 5 up vs 2 |
| like 1///GABA(A) receptors associated | GABARAPL3 | | |
| protein lik | | | |
| gametogenetin binding protein 2 | GGNBP2 | 1.04838 | 5 up vs 2 |
| GNAS complex locus | GNAS | −1.02845 | 5 down vs 2 |
| G protein-coupled receptor 109B | GPR109B | −1.16922 | 5 down vs 2 |
| heparin-binding EGF-like growth factor | HBEGF | −1.06958 | 5 down vs 2 |
| heparin-binding EGF-like growth factor | HBEGF | −1.04659 | 5 down vs 2 |
| heterogeneous nuclear ribonucleoprotein | HNRNPA1/// | 1.04636 | 5 up vs 2 |
| A1///hypothetical LOC100506653 | LOC100506653 | | |
| heat shock 70 kDa protein 14 | HSPA14 | −1.07043 | 5 down vs 2 |
| inhibitor of DNA binding 1, dominant | ID1 | −1.21144 | 5 down vs 2 |
| negative helix-loop-helix protein | | | |
| inhibitor of DNA binding 2, dominant | ID2///ID2B | 1.06142 | 5 up vs 2 |
| negative helix-loop-helix protein/// | | | |
| inhibitor of | | | |
| interferon induced with helicase C | IFIH1 | −1.03516 | 5 down vs 2 |
| domain 1 | | | |
| interferon, gamma | IFNG | 1.37881 | 5 up vs 2 |
| interleukin 8 | IL8 | −1.98785 | 5 down vs 2 |
| interleukin 8 | IL8 | −1.90769 | 5 down vs 2 |
| inhibitor of growth family, member 3 | ING3 | −1.07702 | 5 down vs 2 |
| importin 11///leucine rich repeat | IPO11/// | 1.09786 | 5 up vs 2 |
| containing 70 | LRRC70 | | |
| inositol 1,4,5-trisphosphate 3-kinase B | ITPKB | −1.06792 | 5 down vs 2 |
| influenza virus NS 1A binding protein | IVNS1ABP | 1.09316 | 5 up vs 2 |
| jumonji domain containing 6 | JMJD6 | 1.02649 | 5 up vs 2 |
| jumonji domain containing 6 | JMJD6 | 1.07983 | 5 upvs 2 |
| junction mediating and regulatory protein, | JMY | 1.10319 | 5 up vs 2 |
| p53 cofactor | | | |
| jun D proto-oncogene | JUND | −1.00983 | 5 down vs 2 |
| jun D proto-oncogene | JUND | −1.00507 | 5 down vs 2 |
| kelch repeat and BTB (POZ) domain | KBTBD6 | 1.1573 | 5 up vs 2 |
| containing 6 | | | |
| potassium inwardly-rectifying channel, | KCNJ2 | −1.11538 | 5 down vs 2 |
| subfamily J, member 2 | | | |
| Kruppel-like factor 6 | KLF6 | −1.12771 | 5 down vs 2 |
| Kruppel-like factor 6 | KLF6 | −1.05918 | 5 down vs 2 |
| Kruppel-like factor 6 | KLF6 | −1.02811 | 5 down vs 2 |
| Kruppel-like factor 6 | KLF6 | −1.01155 | 5 down vs 2 |
| Kruppel-like factor 9 | KLF9 | 1.32208 | 5 up vs 2 |
| Kruppel-like factor 9 | KLF9 | 1.32901 | 5 up vs 2 |
| kelch-like 15 (Drosophila) | KLHL15 | 1.14559 | 5 up vs 2 |
| v-Ki-ras2 Kirsten rat sarcoma viral | KRAS | −1.09233 | 5 down vs 2 |
| oncogene homolog | | | |
| lymphocyte cytosolic protein 2 (SH2 | LCP2 | 1.01988 | 5 up vs 2 |
| domain containing leukocyte protein of | | | |
| 76 kDa) | | | |
| Leucine rich repeat (in FLII) interacting | LRRFIP1 | 1.1931 | 5 up vs 2 |
| protein 1 | | | |
| v-maf musculoaponeurotic fibrosarcoma | MAFF | 1.06254 | 5 up vs 2 |
| oncogene homolog F (avian) | | | |
| v-maf musculoaponeurotic fibrosarcoma | MAFF | 1.20496 | 5 up vs 2 |
| oncogene homolog F (avian) | | | |
| muscleblind-like (Drosophila) | MBNL1 | 1.00206 | 5 up vs 2 |
| mediator complex subunit 6 | MED6 | 1.14196 | 5 up vs 2 |
| mex-3 homolog C (C. elegans) | MEX3C | 1.01582 | 5 up vs 2 |
| mex-3 homolog C (C. elegans) | MEX3C | 1.03488 | 5 up vs 2 |
| mesoderm induction early response 1 | MIER1 | 1.00932 | 5 up vs 2 |
| homolog (Xenopus laevis) | | | |
| MOP-1 | MOP-1 | −1.12436 | 5 down vs 2 |
| M-phase phosphoprotein 6 | MPHOSPH6 | −1.00988 | 5 down vs 2 |
| metastasis suppressor 1 | MTSS1 | −1.05163 | 5 down vs 2 |
| myosin regulatory light chain interacting | MYLIP | 1.21045 | 5 up vs 2 |
| protein | | | |
| myosin regulatory light chain interacting | MYLIP | 1.21533 | 5 up vs 2 |
| protein | | | |
| nucleosome assembly protein 1-like 5 | NAP1L5 | 1.21367 | 5 up vs 2 |
| NADH dehydrogenase (ubiquinone) 1 | NDUFA10 | −1.0398 | 5 down vs 2 |
| alpha subcomplex, 10, 42 kDa | | | |
| nuclear factor, interleukin 3 regulated | NFIL3 | −1.02408 | 5 down vs 2 |
| nuclear factor of kappa light polypeptide | NFKBIA | −1.18247 | 5 down vs 2 |
| gene enhancer in B−cells inhibitor, alpha | | | |
| nuclear factor of kappa light polypeptide | NFKBIZ | −1.09681 | 5 down vs 2 |
| gene enhancer in B-cells inhibitor, zeta | | | |
| nuclear factor of kappa light polypeptide | NFKBIZ | −1.06015 | 5 down vs 2 |
| gene enhancer in B-cells inhibitor, zeta | | | |
| nuclear transcription factor Y, alpha | NFYA | −1.06624 | 5 down vs 2 |
| NLR family, pyrin domain containing 3 | NLRP3 | −1.07131 | 5 down vs 2 |
| nuclear receptor subfamily 1, group D, | NR1D1/// | 1.08102 | 5 up vs 2 |
| member 1///thyroid hormone receptor, | THRA | | |
| alpha (er | | | |
| nuclear receptor subfamily 4, group A, | NR4A2 | 1.05002 | 5 up vs 2 |
| member 2 | | | |
| nuclear receptor subfamily 4, group A, | NR4A2 | 1.07373 | 5 up vs 2 |
| member 2 | | | |
| nuclear receptor subfamily 4, group A, | NR4A2 | 1.08247 | 5 up vs 2 |
| member 2 | | | |
| NTF2-like export factor 1 | NXT1 | 1.04874 | 5 up vs 2 |
| oncostatin M | OSM | 1.08465 | 5 up vs 2 |
| PDX1 C-terminal inhibiting factor 1 | PCIF1 | −1.04054 | 5 down vs 2 |
| PDX1 C-terminal inhibiting factor 1 | PCIF1 | −1.02634 | 5 down vs 2 |
| programmed cell death 4 (neoplastic | PDCD4 | 1.02227 | 5 up vs 2 |
| transformation inhibitor) | | | |
| period homolog 1 (Drosophila) | PER1 | 1.03653 | 5 up vs 2 |
| peroxisomal biogenesis factor 12 | PEX12 | 1.02217 | 5 up vs 2 |
| phospholipase A1 member A | PLA1A | −1.01471 | 5 down vs 2 |
| polo-like kinase 3 | PLK3 | 1.13892 | 5 up vs 2 |
| protein phosphatase 1, regulatory | PPP1R15A | −1.1735 | 5 down vs 2 |
| (inhibitor) subunit 15A | | | |
| protein phosphatase 1, regulatory | PPP1R15A | −1.1346 | 5 down vs 2 |
| (inhibitor) subunit 15A | | | |
| protein phosphatase 1, regulatory | PPP1R3B | −1.12317 | 5 down vs 2 |
| (inhibitor) subunit 3B | | | |
| protein phosphatase 1, regulatory | PPP1R3D | −1.09914 | 5 down vs 2 |
| (inhibitor) subunit 3D | | | |
| protein phosphatase 2, regulatory subunit | PPP2R5C | 1.23968 | 5 up vs 2 |
| B′, gamma | | | |
| protein kinase, cAMP-dependent, | PRKAR1A | −1.03818 | 5 down vs 2 |
| regulatory, type I, alpha (tissue specific | | | |
| extinguisher | | | |
| proteasome (prosome, macropain) 26S | PSMD12 | 1.00539 | 5 up vs 2 |
| subunit, non-ATPase, 12 | | | |
| pentraxin 3, long | PTX3 | −1.15434 | 5 down vs 2 |
| RAN binding protein 2///RANBP2-like | RANBP2/// | 1.17929 | 5 up vs 2 |
| and GRIP domain containing 1/// | RGPD1/// | | |
| RANBP2-like and | RGPD2/// | | |
| | RGPD3/// | | |
| | RGPD4/// | | |
| | RGPD5/// | | |
| | RGPD6/// | | |
| | RGPD8 | | |
| RasGEF domain family, member 1B | RASGEF1B | −1.25081 | 5 down vs 2 |
| retinoblastoma binding protein 6 | RBBP6 | −1.11118 | 5 down vs 2 |
| RNA binding motif protein 8A | RBM8A | −1.14395 | 5 down vs 2 |
| ring finger and CCCH-type domains 2 | RC3H2 | −1.06967 | 5 down vs 2 |
| ring finger and CCCH-type domains 2 | RC3H2 | 1.04022 | 5 up vs 2 |
| regulator of G-protein signaling 1 | RGS1 | 1.43216 | 5 up vs 2 |
| regulator of G-protein signaling 1 | RGS1 | 1.4488 | 5 up vs 2 |
| ras homolog gene family, member H | RHOH | 1.2117 | 5 up vs 2 |
| ring finger protein 103 | RNF103 | −1.21823 | 5 down vs 2 |
| ribosomal protein S16 pseudogene 5 | RPS16P5 | 1.18621 | 5 up vs 2 |
| ribosomal protein S27 | RPS27 | 1.09849 | 5 up vs 2 |
| RNA polymerase I transcription factor | RRN3P2 | −1.00927 | 5 down vs 2 |
| homolog (S. cerevisiae) pseudogene 2 | | | |
| Shwachman-Bodian-Diamond syndrome/// | SBDS/// | 1.05523 | 5 up vs 2 |
| Shwachman-Bodian-Diamond | SBDSP1 | | |
| syndrome pseudogene 1 | | | |
| Shwachman-Bodian-Diamond syndrome/// | SBDS/// | 1.13477 | 5 up vs 2 |
| Shwachman-Bodian-Diamond | SBDSP1 | | |
| syndrome pseudogene 1 | | | |
| splicing factor 1 | SF1 | −1.01311 | 5 down vs 2 |
| splicing factor, arginine/serine-rich 15 | SFRS15 | 1.10596 | 5 up vs 2 |
| salt-inducible kinase 1 | SIK1 | 1.12586 | 5 up vs 2 |
| solute carrier family 35, member F5 | SLC35F5 | 1.01234 | 5 up vs 2 |
| solute carrier family 6 (neurotransmitter | SLC6A6 | 1.00355 | 5 up vs 2 |
| transporter, taurine), member 6 | | | |
| superoxide dismutase 2, mitochondrial | SOD2 | −1.22325 | 5 down vs 2 |
| SON DNA binding protein | SON | 1.02302 | 5 up vs 2 |
| splicing regulatory glutamine/lysine-rich | SREK1 | 1.07951 | 5 up vs 2 |
| protein 1 | | | |
| slingshot homolog 2 (Drosophila) | SSH2 | −1.12199 | 5 down vs 2 |
| serine/threonine kinase 17b | STK17B | −1.04782 | 5 down vs 2 |
| STT3, subunit of the | STT3B | 1.20119 | 5 up vs 2 |
| oligosaccharyltransfemse complex, | | | |
| homolog B (S. cerevisiae) | | | |
| SYS1 Golgi-localized integral membrane | SYS1 | 1.09265 | 5 up vs 2 |
| protein homolog (S. cerevisiae) | | | |
| T-cell activation RhoGTPase activating | TAGAP | 1.06005 | 5 up vs 2 |
| protein | | | |
| T-cell activation RhoGTPase activating | TAGAP | 1.10437 | 5 up vs 2 |
| protein | | | |
| TCDD-inducible poly(ADP-ribose) | TIPARP | 1.07912 | 5 up vs 2 |
| polymerase | | | |
| transketolase-like 1 | TKTL1 | 1.16307 | 5 up vs 2 |
| transmembrane emp24 protein transport | TMED5 | 1.18868 | 5 up vs 2 |
| domain containing 5 | | | |
| transmembrane protein 107 | TMEM107 | 1.00863 | 5 up vs 2 |
| tumor necrosis factor, alpha-induced | TNFAIP3 | 1.26723 | 5 up vs 2 |
| protein 3 | | | |
| tumor necrosis factor, alpha-induced | TNFAIP3 | 1.37887 | 5 up vs 2 |
| protein 3 | | | |
| tropomyosin 3 | TPM3 | −1.18791 | 5 down vs 2 |
| tropomyosin 3 | TPM3 | 1.0158 | 5 upvs 2 |
| transformer 2 beta homolog (Drosophila) | TRA2B | 1.02077 | 5 up vs 2 |
| transformer 2 beta homolog (Drosophila) | TRA2B | 1.02181 | 5 up vs 2 |
| tribbles homolog 1 (Drosophila) | TRIB1 | −1.26438 | 5 down vs 2 |
| tripartite motif-containing 11 | TRIM11 | 1.01083 | Sup vs 2 |
| T5C22 domain family, member 2 | TSC22D2 | −1.13212 | 5 down vs 2 |
| ubiquitin-conjugating enzyme E2D3 | UBE2D3 | 1.0969 | 5 up vs 2 |
| (UBC4/5 homolog, yeast) | | | |
| vimentin | VIM | 1.14777 | 5 upvs 2 |
| WD repeat domain 5B | WDR5B | −1.04664 | 5 down vs 2 |
| WAS/WASL interacting protein family, | WIPF1 | −1.09213 | 5 down vs 2 |
| member 1 | | | |
| YME1-like 1(S. cerevisiae) | YME1L1 | 1.05139 | 5 up vs 2 |
| yrdC domain containing (E. coli) | YRDC | −1.01475 | 5 down vs 2 |
| zinc finger and BTB domain containing 10 | ZBTB10 | 1.00536 | 5 up vs 2 |
| zinc finger and BTB domain containing 10 | ZBTB10 | 1.00543 | 5 up vs 2 |
| zinc finger and BTB domain containing 10 | ZBTB10 | 1.00892 | 5 up vs 2 |
| zinc finger and BTB domain containing 11 | ZBTB11 | −1.05711 | 5 down vs 2 |
| zinc finger and BTB domain containing 16 | ZBTB16 | 1.23945 | 5 up vs 2 |
| zinc finger and BTB domain containing 24 | ZBTB24 | −1.10338 | 5 down vs 2 |
| zinc finger and BTB domain containing 25 | ZBTB25 | 1.14699 | 5 up vs 2 |
| zinc finger and BTB domain containing 3 | ZBTB3 | −1.01121 | 5 down vs 2 |
| zinc finger and BTB domain containing 6 | ZBTB6 | 1.00105 | 5 up vs 2 |
| zinc finger CCCH-type containing 12A | ZC3H12A | 1.06066 | 5 up vs 2 |
| zinc finger, AN1-type domain 2A | ZFAND2A | −1.03034 | 5 down vs 2 |
| zinc finger, C3H1-type containing | ZFC3H1 | 1.0328 | 5 up vs 2 |
| zinc finger protein 36, C3H type, | ZFP36 | −1.30242 | 5 down vs 2 |
| homolog (mouse) | | | |
| zinc finger, MYM-type 2 | ZMYM2 | −1.14017 | 5 down vs 2 |
| zinc finger protein 12 | ZNF12 | 1.02677 | 5 up vs 2 |
| zinc finger protein 394 | ZNF394 | 1.0536 | 5 up vs 2 |
| zinc finger protein 780A | ZNF780A | −1.00667 | 5 down vs 2 |

### Example 2: Twin Study

Experiments were conducted using methodology described in Example 1, but specific to two individuals, of which one was a twin discordant for concussion and her monozygotic twin sibling serving as a control.

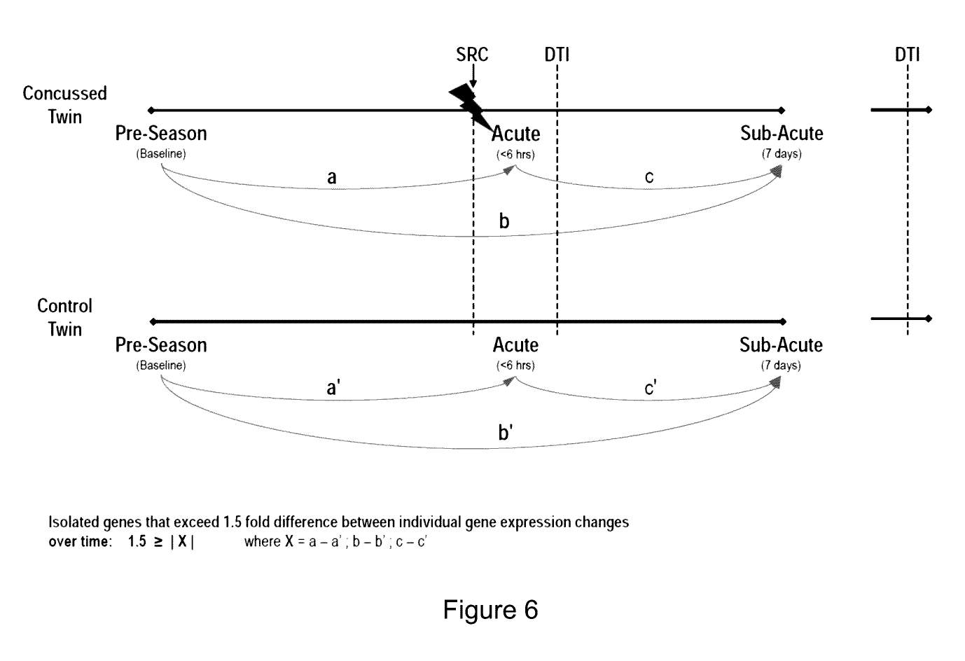

A comparison of monozygotic twins discordant for concussion controls for genetic variance and reduces variance due to environmental circumstances, thus serving to highlight differences due to phenotypic-related variables. One such pair of twins were assessed to determine acute and sub-acute changes in global gene expression in peripheral leukocytes before and after sports-related concussion. The difference in the gene expression level changes between each time point—baseline (T1), acutely post-SRC (T2), and sub-acutely post-SRC (T5) was determined for all gene probes for the concussed twin and control twin. Then, the difference between the twins was assessed by calculating the difference in the observed changes (e.g., the difference between T2−T1 of concussed twin and T2−T1 of control twin). The study design is depicted in FIG. 6.

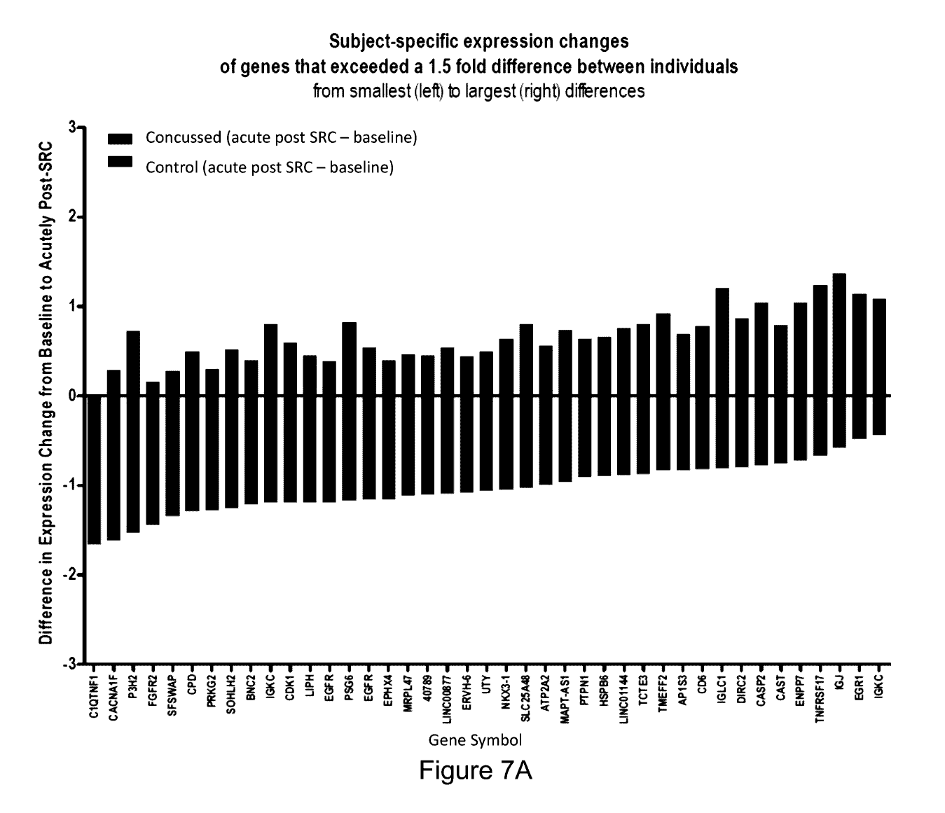

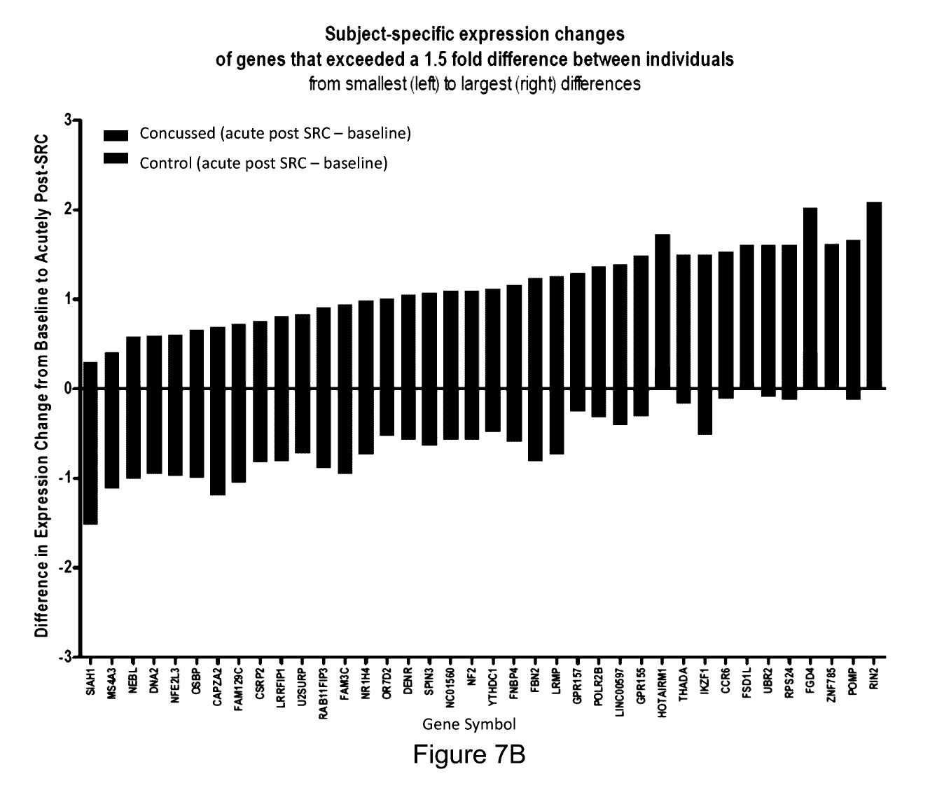

The results of these studies are shown in FIG. 7A-FIG. 7B, and Table 7A Table 7B, Table 8A, Table 8B, Table 9A, and Table 9B. Specifically, Table 7A Table 7B, Table 8A, Table 8B, Table 9A, and Table 9B depict the subject-specific gene expression changes of gene probes that exceeded a 1.5-fold difference between concussed and non-concussed twin.

A list of gene transcripts observed to be upregulated in the concussed twin relative to the control twin at 6 hours post SRC (T2) compared to baseline (T1) is shown in Table 7A. A list of gene transcripts observed to be downregulated in the concussed twin relative to the control twin at 6 hours post SRC (T2) compared to baseline (T1) is shown in Table 7B.

A list of gene transcripts observed to be upregulated in the concussed twin relative to the control twin at 7 days post SRC (T5) compared to baseline (T1) is shown in Table 8A. A list of gene transcripts observed to be downregulated in the concussed twin relative to the control twin at 7 days post SRC (T5) compared to baseline (T1) is shown in Table 8B.

A list of gene transcripts observed to be upregulated in the concussed twin relative to the control twin at 7 days post SRC (T5) compared to 6 hours post SRC (T2) is shown in Table 9A. A list of gene transcripts observed to be downregulated in the concussed twin relative to the control twin at 7 days post SRC (T5) compared to 6 hours post SRC (T2) is shown in Table 9B.

TABLE 7A
Up-regulated differentially expressed transcripts of concussed twin relative to
control twin at 6 hours post SRC (T2) vs. Baseline (T1)

| | | | | Difference |
|---|---|---|---|---|
| | | Concussed | Control | (Concussed |
| | | Fold | Fold | Fold Change- |
| | Gene | Change | Change | Control Fold |
| Gene Name | Symbol | (T2 vs. T1) | (T2 vs. T1) | Change) |
| leucine rich repeat (in FLII) | LRRFIP1 | 0.81 | −0.80 | 1.61 |
| interacting protein 1 | | | | |
| polymerase (RNA) II (DNA directed) | POLR2B | 1.37 | −0.31 | 1.67 |
| polypeptide B, 140 kDa | | | | |
| family with sequence similarity 129, | FAM129C | 0.72 | −1.05 | 1.77 |
| member C | | | | |
| G protein-coupled receptor 155 | GPR155 | 1.49 | −0.30 | 1.79 |
| long intergenic non-protein coding | LINC00597 | 1.39 | −0.40 | 1.79 |
| RNA 597 | | | | |
| family with sequence similarity 3, | FAM3C | 0.94 | −0.94 | 1.88 |
| member C | | | | |
| Ras and Rab interactor 2 | RIN2 | 2.13 | 0.09 | 2.04 |
| HOXA transcript antisense RNA, | HOTAIRM1 | 1.74 | 0.23 | 1.50 |
| myeloid-specific 1 | | | | |
| olfactory receptor, family 7, | OR7D2 | 1.01 | −0.52 | 1.52 |
| subfamily D, member 2 | | | | |
| membrane-spanning 4-domains, | MS4A3 | 0.41 | −1.11 | 1.53 |
| subfamily A, member 3 | | | | |
| (hematopoietic cell-specific) | | | | |
| DNA replication helicase/nuclease 2 | DNA2 | 0.59 | −0.94 | 1.53 |
| fibronectin type III and SPRY | FSD1L | 1.60 | 0.07 | 1.54 |
| domain containing 1-like | | | | |
| G protein-coupled receptor 157 | GPR157 | 1.29 | −0.25 | 1.54 |
| U2 snRNP-associated SURP domain | U2SURP | 0.83 | −0.72 | 1.55 |
| containing | | | | |
| cysteine and glycine-rich protein 2 | CSRP2 | 0.75 | −0.82 | 1.56 |
| nuclear factor, erythroid 2-like 3 | NFE2L3 | 0.60 | −0.97 | 1.57 |
| nebulette | NEBL | 0.58 | −1.00 | 1.58 |
| YTH domain containing 1 | YTHDC1 | 1.12 | −0.48 | 1.60 |
| density-regulated protein | DENR | 1.05 | −0.56 | 1.60 |
| FYVE, RhoGEF and PH domain | FGD4 | 2.02 | 0.40 | 1.62 |
| containing 4 | | | | |
| zinc finger protein 785 | ZNF785 | 1.62 | 0.00 | 1.63 |
| chemokine (C-C motif) receptor 6 | CCR6 | 1.53 | −0.11 | 1.64 |
| oxysterol binding protein | OSBP | 0.66 | −0.99 | 1.65 |
| long intergenic non-protein coding | LINC01560 | 1.09 | −0.56 | 1.65 |
| RNA 1560 | | | | |
| thyroid adenom aassociated | THADA | 1.50 | −0.16 | 1.65 |
| neurofibromin 2 (merlin) | NF2 | 1.09 | −0.57 | 1.66 |
| ubiquitin protein ligase E3 | UBR2 | 1.61 | −0.09 | 1.70 |
| component n-recognin 2 | | | | |
| spindlin family, member 3 | SPIN3 | 1.07 | −0.63 | 1.70 |
| nuclear receptor subfamily 1, group | NR1H4 | 0.98 | −0.73 | 1.71 |
| H, member 4 | | | | |
| ribosomal protein S24 | RPS24 | 1.61 | −0.12 | 1.72 |
| formin binding protein 4 | FNBP4 | 1.16 | −0.59 | 1.74 |
| RAB11 family interacting protein 3 | RAB11FIP3 | 0.91 | −0.88 | 1.78 |
| (class II) | | | | |
| proteasome maturation protein | POMP | 1.66 | −0.12 | 1.79 |
| siah E3 ubiquitin protein ligase 1 | SIAH1 | 0.30 | −1.51 | 1.81 |
| capping protein (actin filament) | CAPZA2 | 0.69 | −1.19 | 1.87 |
| muscle Z-line, alpha 2 | | | | |
| lymphoid-restricted membrane | LRMP | 1.26 | −0.73 | 1.99 |
| protein | | | | |
| IKAROS family zinc finger 1 | IKZF1 | 1.50 | −0.51 | 2.01 |
| (Ikaros) | | | | |
| fibrillin 2 | FBN2 | 1.24 | −0.81 | 2.06 |

TABLE 7B
Down-regulated differentially expressed transcripts of concussed twin relative
to control twin at 6 hours post SRC (T2) vs. Baseline (T1)

| | | | | Difference |
|---|---|---|---|---|
| | | Concussed | Control | (Concussed |
| | | Fold | Fold | Fold Change- |
| | Gene | Change | Change | Control Fold |
| Gene Name | Symbol | (T2 vs. T1) | (T2 vs. T1) | Change) |
| prolyl 3-hydroxylase 2 | P3H2 | −1.53 | 0.72 | −2.25 |
| immunoglobulin kappa constant | IGKC | −1.19 | 0.80 | −1.99 |
| tumor necrosis factor receptor | TNFRSF17 | −0.66 | 1.23 | −1.90 |
| superfamily, member 17 | | | | |
| caspase 2, apoptosis-related cysteine | CASP2 | −0.77 | 1.04 | −1.81 |
| peptidase | | | | |
| spermatogenesis and oogenesis | SOHLH2 | −1.25 | 0.52 | −1.77 |
| specific basic helix-loop-helix 2 | | | | |
| transmembrane protein with EGF- | TMEFF2 | −0.83 | 0.92 | −1.75 |
| like and two follistatin-like domains 2 | | | | |
| epidermal growth factor receptor | EGFR | −1.15 | 0.54 | −1.69 |
| epidermal growth factor receptor | EGFR | −1.19 | 0.38 | −1.57 |
| immunoglobulin lambda constant 1 | IGLC1 | −0.80 | 1.20 | −2.00 |
| (Mcg marker) | | | | |
| pregnancy specific beta-1- | PSG6 | −1.17 | 0.82 | −2.00 |
| glycoprotein 6 | | | | |
| immunoglobulin J polypeptide, linker | IGJ | −0.58 | 1.37 | −1.95 |
| protein for immunoglobulin alpha | | | | |
| and mu polypeptides | | | | |
| calcium channel, voltage-dependent, | CACNA1F | −1.61 | 0.29 | −1.90 |
| L type, alpha 1F subunit | | | | |
| solute carrier family 25, member 48 | SLC25A48 | −1.02 | 0.80 | −1.83 |
| carboxypeptidase D | CPD | −1.29 | 0.49 | −1.78 |
| cyclin-dependent kinase 1 | CDK1 | −1.19 | 0.59 | −1.78 |
| ectonucleotide | ENPP7 | −0.72 | 1.04 | −1.76 |
| pyrophosphatase/phosphodiesterase 7 | | | | |
| MAPT antisense RNA 1 | MAPT-AS1 | −0.96 | 0.73 | −1.69 |
| NK3 homeobox 1 | NKX3-1 | −1.04 | 0.63 | −1.67 |
| t-complex-associated-testis-expressed 3 | TCTE3 | −0.87 | 0.80 | −1.66 |
| disrupted in renal carcinoma 2 | DIRC2 | −0.79 | 0.87 | −1.65 |
| lipase, member H | LIPH | −1.19 | 0.45 | −1.63 |
| long intergenic non-protein coding | LINC00877 | −1.09 | 0.54 | −1.63 |
| RNA 877 | | | | |
| long intergenic non-protein coding | LINC01144 | −0.88 | 0.75 | −1.63 |
| RNA 1144 | | | | |
| early growth response 1 | EGR1 | −0.48 | 1.14 | −1.62 |
| splicing factor, suppressor of white- | SFSWAP | −1.34 | 0.28 | −1.62 |
| apricot family | | | | |
| C1q and tumor necrosis factor related | C1QTNF1 | −1.66 | -0.04 | −1.62 |
| protein 1 | | | | |
| basonuclin 2 | BNC2 | −1.21 | 0.40 | −1.61 |
| CD6 molecule | CD6 | −0.82 | 0.78 | −1.60 |
| fibroblast growth factor receptor 2 | FGFR2 | −1.44 | 0.16 | −1.60 |
| protein kinase, cGMP-dependent, | PRKG2 | −1.27 | 0.30 | −1.57 |
| type II | | | | |
| mitochondrial ribosomal protein L47 | MRPL47 | −1.11 | 0.46 | −1.57 |
| ATPase, Ca++ transporting, cardiac | ATP2A2 | −0.99 | 0.56 | −1.56 |
| muscle, slow twitch 2 | | | | |
| septin 4 | 40789 | −1.10 | 0.45 | −1.55 |
| ubiquitously transcribed | UTY | −1.06 | 0.49 | −1.54 |
| tetratricopeptide repeat containing, | | | | |
| Y-linked | | | | |
| epoxide hydrolase 4 | EPHX4 | −1.15 | 0.40 | −1.54 |
| heat shock protein, alpha-crystallin- | HSPB6 | −0.89 | 0.66 | −1.54 |
| related, B6 | | | | |
| calpastatin | CAST | −0.75 | 0.79 | −1.54 |
| protein tyrosine phosphatase, non- | PTPN1 | −0.90 | 0.64 | −1.53 |
| receptor type 1 | | | | |
| endogenous retrovirus group H, | ERVH-6 | −1.08 | 0.44 | −1.52 |
| member 6 | | | | |
| adaptor-related protein complex 1, | AP1S3 | −0.83 | 0.69 | −1.51 |
| sigma 3 subunit | | | | |
| immunoglobulin kappa constant | IGKC | −0.43 | 1.08 | −1.51 |

TABLE 8A
Up-regulated differentially expressed transcripts of concussed twin relative to
control twin at 7 days post SRC (T5) vs. Baseline (T1)

| | | | | Difference |
|---|---|---|---|---|
| | | Concussed | Control | (Concussed |
| | | Fold | Fold | Fold Change- |
| | Gene | Change | Change | Control Fold |
| Gene Name | Symbol | (T5 vs. T1) | (T5 vs. T1) | Change) |
| leucine rich repeat (in FLII) | LRRFIP1 | 1.03 | −0.53 | 1.56 |
| interacting protein 1 | | | | |
| polymerase (RNA) II (DNA directed) | POLR2B | 2.66 | 0.71 | 1.95 |
| polypeptide B, 140 kDa | | | | |
| family with sequence similarity 129, | FAM129C | 1.03 | −0.51 | 1.54 |
| member C | | | | |
| G protein-coupled receptor 155 | GPR155 | 1.64 | −0.49 | 2.12 |
| long intergenic non-protein coding | LINC00597 | 1.10 | −0.71 | 1.81 |
| RNA 597 | | | | |
| family with sequence similarity 3, | FAM3C | 1.08 | −0.79 | 1.87 |
| member C | | | | |
| Ras and Rab interactor 2 | RIN2 | 1.50 | −0.22 | 1.72 |
| exportin 7 | XPO7 | 0.78 | −0.89 | 1.67 |
| protein tyrosine phosphatase, | PTPRC | 0.96 | −0.62 | 1.58 |
| receptor type, C | | | | |
| epiregulin | EREG | 0.01 | −1.72 | 1.73 |
| ataxin 1 | ATXN1 | 0.29 | −1.37 | 1.65 |
| cytoplasmic polyadenylation element | CPEB4 | 0.75 | −0.80 | 1.54 |
| binding protein 4 | | | | |
| proteasome (prosome, macropain) | PSMB7 | 0.66 | −1.01 | 1.68 |
| subunit, beta type, 7 | | | | |
| transmembrane protein 229B | TMEM229B | 0.97 | −0.68 | 1.66 |
| adhesion G protein-coupled receptor G1 | ADGRG1 | 0.10 | −1.41 | 1.50 |
| pyrin and HIN domain family, member 1 | PYHIN1 | −0.19 | −1.72 | 1.53 |
| neuregulin 1 | NRG1 | 1.68 | 0.14 | 1.54 |
| phospholipase D1, | PLD1 | 1.44 | −0.13 | 1.56 |
| phosphatidylcholine-specific | | | | |
| Rho-related BTB domain containing 3 | RHOBTB3 | 0.95 | −0.90 | 1.85 |
| DDB1 and CUL4 associated factor 17 | DCAF17 | 1.05 | −0.52 | 1.57 |
| zinc finger protein 578 | ZNF578 | 0.19 | −1.33 | 1.52 |
| U2 snRNP-associated SURP domain | U2SURP | 0.36 | −1.28 | 1.64 |
| containing | | | | |
| MCM3AP antisense RNA 1 | MCM3AP- | 0.87 | −0.67 | 1.54 |
| | AS1 | | | |
| ELK4, ETS-domain protein | ELK4 | 0.77 | −1.04 | 1.81 |
| (SRF accessory protein 1) | | | | |
| glutamate-cysteine ligase, modifier | GCLM | 1.57 | −0.18 | 1.75 |
| subunit | | | | |
| Kruppel-like factor 12 | KLF12 | 0.53 | −1.32 | 1.85 |
| tumor necrosis factor receptor | TNFRSF25 | 0.54 | −1.03 | 1.57 |
| superfamily, member 25 | | | | |
| ectonucleotide | ENPP5 | 0.75 | −0.80 | 1.56 |
| pyrophosphatase/phosphodiesterase 5 | | | | |
| (putative) | | | | |
| killer cell lectin-like receptor | KLRC4 | −0.27 | −1.96 | 1.68 |
| subfamily C, member 4 | | | | |
| ectonucleotide | ENPP5 | 1.03 | −0.65 | 1.68 |
| pyrophosphatase/phosphodiesterase 5 | | | | |
| (putative) | | | | |
| long intergenic non-protein coding | LINC01578 | 0.60 | −1.12 | 1.72 |
| RNA 1578 | | | | |
| mechanistic target of rapamycin | MTOR | 0.37 | −1.14 | 1.51 |
| (serine/threonine kinase) | | | | |
| protein tyrosine phosphatase, non- | PTPN4 | 1.04 | −0.76 | 1.80 |
| receptor type 4 (megakaryocyte) | | | | |
| CD24 molecule | CD24 | 0.64 | −0.87 | 1.51 |
| NEDD4 binding protein 2-like 2 | N4BP2L2 | 1.18 | −0.53 | 1.70 |
| pyrophosphatase (inorganic) 2 | PPA2 | 1.26 | −0.35 | 1.61 |
| PEST proteolytic signal containing | PCNP | 0.11 | −1.60 | 1.71 |
| nuclear protein | | | | |
| ARP2 actin-related protein 2 | ACTR2 | 1.29 | −0.35 | 1.64 |
| homolog (yeast) | | | | |
| Rho-related BTB domain containing 3 | RHOBTB3 | 1.32 | −0.19 | 1.51 |
| lysozyme G-like 2 | LYG2 | 0.90 | −0.68 | 1.57 |
| synaptotagmin-like 3 | SYTL3 | 1.47 | −0.06 | 1.53 |
| mitochondrial ribosomal protein L19 | MRPL19 | 1.72 | −0.41 | 2.13 |
| metastasis associated lung | MALAT1 | 0.78 | −0.80 | 1.59 |
| adenocarcinoma transcript 1 (non- | | | | |
| protein coding) | | | | |
| nicotinamide nucleotide | NMNAT3 | 0.61 | −1.16 | 1.77 |
| adenylyltransferase 3 | | | | |
| TAF15 RNA polymerase II, TATA | TAF15 | 1.32 | −0.29 | 1.62 |
| box binding protein (TBP)-associated | | | | |
| factor, 68 kDa | | | | |
| platelet factor 4 variant 1 | PF4V1 | 0.99 | −0.61 | 1.60 |
| RUN and FYVE domain containing 2 | RUFY2 | 1.41 | −0.45 | 1.87 |
| sperm associated antigen 1 | SPAG1 | 0.83 | −0.69 | 1.52 |
| transcription factor 7-like 2 | TCF7L2 | 1.62 | −0.22 | 1.84 |
| (T-cell specific, HMG-box) | | | | |
| protein tyrosine phosphatase, | PTPRC | 1.23 | −0.48 | 1.71 |
| receptor type, C | | | | |

TABLE 8B
Down-regulated differentially expressed transcripts of concussed twin relative
to control twin at 7 days post SRC (T5) vs. Baseline (T1)

| | | | | Difference |
|---|---|---|---|---|
| | | Concussed | Control | (Concussed |
| | | Fold | Fold | Fold Change- |
| | Gene | Change | Change | Control Fold |
| Gene Name | Symbol | (T5 vs. T1) | (T5 vs. T1) | Change) |
| prolyl 3-hydroxylase 2 | P3H2 | −1.25 | 0.76 | −2.01 |
| immunoglobulin kappa constant | IGKC | −1.12 | 1.07 | −2.19 |
| tumor necrosis factor receptor | TNFRSF17 | −0.61 | 0.92 | −1.54 |
| superfamily, member 17 | | | | |
| caspase 2, apoptosis-related cysteine | CASP2 | −0.51 | 1.12 | −1.63 |
| peptidase | | | | |
| spermatogenesis and oogenesis | SOHLH2 | −1.82 | −0.02 | −1.81 |
| specific basic helix-loop-helix 2 | | | | |
| transmembrane protein with EGF- | TMEFF2 | −1.81 | 0.99 | −2.80 |
| like and two follistatin-like domains 2 | | | | |
| epidermal growth factor receptor | EGFR | −1.35 | 0.56 | −1.91 |
| epidermal growth factor receptor | EGFR | −2.07 | 0.34 | −2.41 |
| pleckstrin homology-like domain, | PHLDB3 | −0.90 | 0.68 | −1.59 |
| family B, member 3 | | | | |
| mitotic spindle organizing protein 1 | MZT1 | −0.48 | 1.07 | −1.55 |
| sialophorin | SPN | −0.58 | 1.01 | −1.59 |
| ADP-ribosyltransferase 4 | ART4 | −1.15 | 0.37 | −1.51 |
| (Dombrock blood group) | | | | |
| glycine dehydrogenase | GLDC | −0.94 | 0.60 | −1.55 |
| (decarboxylating) | | | | |
| Rh-associated glycoprotein | RHAG | −0.86 | 0.68 | −1.54 |
| RAB30, member RAS oncogene | RAB30 | −1.17 | 0.52 | −1.69 |
| family | | | | |
| ribosomal protein S11 | RPS11 | −1.06 | 0.79 | −1.85 |
| S100 calcium binding protein A8 | S100A8 | −1.17 | 0.67 | −1.84 |
| solute carrier family 22 (organic | SLC22A12 | −0.89 | 0.65 | −1.54 |
| anion/urate transporter), member 12 | | | | |
| long intergenic non-protein coding | LINC01016 | −1.00 | 0.57 | −1.57 |
| RNA 1016 | | | | |
| BicC family RNA binding protein 1 | BICC1 | −0.87 | 0.99 | −1.86 |
| enolase 1, (alpha) | ENO1 | −0.78 | 0.75 | −1.52 |

TABLE 9A
Up-regulated differentially expressed transcripts of concussed twin relative to
control twin at 7 days post SRC (T5) vs. 6 hours post SRC (T2)

| | | | | Difference |
|---|---|---|---|---|
| | | Concussed | Control | (Concussed |
| | | Fold | Fold | Fold Change- |
| | Gene | Change | Change | Control Fold |
| Gene Name | Symbol | (T5 vs. T2) | (T5 vs. T2) | Change) |
| exportin 7 | XPO7 | 0.77 | −0.88 | 1.65 |
| protein tyrosine phosphatase, | PTPRC | 0.61 | −0.94 | 1.55 |
| receptor type, C | | | | |
| epiregulin | EREG | −0.07 | −1.74 | 1.67 |
| ataxin 1 | ATXN1 | 0.39 | −1.11 | 1.50 |
| synaptic vesicle glycoprotein 2B | SV2B | 0.85 | −0.71 | 1.57 |
| glutamine and serine rich 1 | QSER1 | 0.16 | −2.03 | 2.19 |
| InaD-like (Drosophila) | INADL | 0.54 | −0.99 | 1.53 |
| ADAM metallopeptidase with | ADAMTS1 | 1.20 | −0.36 | 1.56 |
| thrombospondin type 1 motif, 1 | | | | |
| myosin, heavy chain 10, non-muscle | MYH10 | 0.75 | −0.77 | 1.52 |
| zinc finger protein 541 | ZNF541 | 0.21 | −1.45 | 1.66 |
| DNA replication and sister chromatid | DSCC1 | 0.58 | −0.98 | 1.55 |
| cohesion 1 | | | | |
| glutamate receptor, ionotropic, N- | GRIN2D | 0.95 | −0.58 | 1.52 |
| methyl D-aspartate 2D | | | | |
| aldo-keto reductase family 1, | AKR1C3 | 1.04 | −0.48 | 1.52 |
| member C3 | | | | |
| PIFl 5′-to-3′ DNA helicase | PIF1 | 0.25 | −1.25 | 1.50 |
| DENN/MADD domain containing 1B | DENND1B | 0.82 | −0.82 | 1.64 |
| poly(rC) binding protein 2 | PCBP2 | 1.50 | −0.42 | 1.92 |
| IKAROS family zinc finger 3 (Aiolos) | IKZF3 | 0.76 | −0.75 | 1.51 |
| general transcription factor EH, | GTF2H5 | 1.29 | −0.33 | 1.62 |
| polypeptide 5 | | | | |
| killer cell lectin-like receptor | KLRB1 | 0.48 | −1.04 | 1.52 |
| subfamily B, member 1 | | | | |
| lysine (K)-specific methyltransferase 2C | KMT2C | 0.73 | −0.87 | 1.60 |
| LIM domain binding 2 | LDB2 | 1.56 | 0.00 | 1.57 |
| ataxin 3 | ATXN3 | 0.79 | −0.75 | 1.54 |
| protease, serine, 23 | PRSS23 | 1.07 | −0.46 | 1.53 |

TABLE 9B
Down-regulated differentially expressed transcripts of concussed twin relative
to control twin at 7 days post SRC (T5) vs. 6 hours post SRC (T2)

| | | | | Difference |
|---|---|---|---|---|
| | | Concussed | Control | (Concussed |
| | | Fold | Fold | Fold Change- |
| | Gene | Change | Change | Control Fold |
| Gene Name | Symbol | (T5 vs. T2) | (T5 vs. T2) | Change) |
| pleckstrin homology-like domain, | PHLDB3 | −0.64 | 0.91 | −1.55 |
| family B, member 3 | | | | |
| mitotic spindle organizing protein 1 | MZT1 | −1.03 | 0.73 | −1.76 |
| dachshund family transcription factor 1 | DACH1 | −1.16 | 0.35 | −1.51 |
| mediator complex subunit 18 | MED18 | −1.12 | 0.42 | −1.53 |
| ceroid-lipofuscinosis, neuronal 8 | CLN8 | −0.75 | 0.76 | −1.50 |
| (epilepsy, progressive with mental | | | | |
| retardation) | | | | |
| peptidyl arginine deiminase, type IV | PADI4 | −1.25 | 0.27 | −1.53 |

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of diagnosing brain injury in a subject who has received a head trauma, the method comprising:
a. detecting the level of at least one biomarker in a first biological sample obtained from the subject at a first time point following head trauma;
b. determining that the level of the at least one biomarker in the first biological sample is different when compared to a control; and
c. determining that the subject has a brain injury when the level of the at least one biomarker in the first biological sample is different when compared to the control;
wherein the at least one biomarker is a gene or gene product listed in Table 4A, Table 4B, Table 5A, Table 5B, Table 6, Table 7A, Table 7B, Table 8A, Table 8B, Table 9A, or Table 9B.

2. The method of claim 1, wherein the control is the level of the at least one biomarker in a biological sample obtained from the subject prior to head trauma.

3. The method of claim 1, wherein the control is the level or change in the level of the at least one biomarker in a control subject or population who have not experienced a head trauma.

4. The method claim of claim 1, wherein the level of the at least one biomarker in the first biological sample is different from the control by more than about 1.5 fold.

5. The method of claim 1, wherein one or more of the at least one biomarker is a gene or gene product listed in Table 4A or Table 7A and the first time point is about 6 hours, and wherein the expression level of the at least one biomarker is increased compared to the control.

6. The method of claim 1, wherein one or more of the at least one biomarker is a gene or gene product listed in Table 4B or Table 7B and the first time point is about 6 hours, and wherein the expression level of the at least one biomarker is decreased compared to the control.

7. The method of claim 1, wherein one or more of the at least one biomarker is a gene or gene product listed in Table 5A or Table 8A and the first time point is about 7 days, and wherein the expression level of the at least one biomarker is increased compared to the control.

8. The method of claim 1, wherein one or more of the at least one biomarker is a gene or gene product listed in Table 5B or Table 8B and the first time point is about 7 days, and wherein the expression level of the at least one biomarker is decreased compared to the control.

9. The method of claim 1, wherein the first biological sample is a peripheral mononuclear blood cell (PMBC).

10. The method of claim 1, further comprising effectuating a brain injury treatment to the subject.

11. A method of diagnosing concussion in a subject who has received a head trauma, the method comprising:
a. detecting the level of at least one biomarker in a first biological sample obtained from the subject at a first time point following head trauma;
b. detecting the level of the at least one biomarker in a second biological sample obtained from the subject at a second time point following head trauma;
c. determining that the level of the at least one biomarker in the second biological sample is different as compared to the level of the at least one biomarker in the first biological sample;
d. determining that the subject has a concussion when the level of the at least one biomarker in the second biological sample is different than the level of the at least one biomarker in the first biological sample; and
wherein the at least one biomarker is a gene or gene product listed in Table 6, Table 9A, or Table 9B.

12. The method of claim 11, wherein the first time point is about 6 hours following head trauma.

13. The method of claim 11, wherein the second time point is about 7 days following head trauma.

14. The method of claim 11, wherein the method further comprises detecting that the difference in the level of the at least one biomarker in the second biological sample as compared to the level of the at least one biomarker in the first biological sample is different relative to a control.

15. The method of claim 11, wherein the first biological sample and second biological sample each comprise a peripheral mononuclear blood cell (PMBC).

16. The method of claim 11, further comprising effectuating a brain injury treatment to the subject.

17. A method of assessing the recovery from brain injury in a subject who has received a head trauma, the method comprising:
a. detecting the level of at least one biomarker in a first biological sample obtained from the subject at a first time point following head trauma;
b. determining that the level of the at least one biomarker in the first biological sample is different as compared to a control; and
c. determining the recovery from brain injury when the level of the at least one biomarker in the first biological sample is significantly different when compared to the control level;
wherein the at least one biomarker is a gene or gene product listed in Table 4A, Table 4B, Table 5A, Table 5B, Table 6, Table 7A, Table 7B, Table 8A, Table 8B, Table 9A, or Table 9B.

18. A method of assessing the recovery of a brain injury in a subject who has received a head trauma, the method comprising:
a. detecting the level of at least one biomarker in a first biological sample obtained from the subject at a first time point following head trauma;
b. detecting the level of the at least one biomarker in a second biological sample obtained from the subject at a second time point following head trauma;
c. determining that the level of the at least one biomarker in the second biological sample is different as compared to the level of the at least one biomarker in the first biological sample; and
d. determining the recovery from brain injury when the level of the at least one biomarker in the second biological sample is significantly different than the level of the at least one biomarker in the first biological sample;
wherein the at least one biomarker is a gene or gene product listed in Table 6, Table 9A, or Table 9B.

19. A method of treating an individual with brain injury comprising administering a brain injury treatment to a subject identified as having a differentially expressed level of at least one biomarker in a biological sample obtained after head trauma, wherein the at least one biomarker is a gene or gene product listed in Table 4A, Table 4B, Table 5A, Table 5B, Table 6, Table 7A, Table 7B, Table 8A, Table 8B, Table 9A, or Table 9B.
